(19) United States
(12) Patent Application Publication
(10) Pub. No.: US 2026/0055109 A1
(43) Pub. Date: Feb. 26, 2026

(54) IBOGAINE COMPOUNDS FOR TREATING BRAIN DISORDERS

(71) Applicant: The Regents of the University of California, Oakland, CA (US)

(72) Inventors: Rishab IYER, Oakland, CA (US); Andras DOMOKOS, Oakland, CA (US); David E. OLSON, Oakland, CA (US)

(21) Appl. No.: 19/077,515

(22) Filed: Mar. 12, 2025

Related U.S. Application Data
(60) Provisional application No. 63/564,092, filed on Mar. 12, 2024.

(51) Int. Cl. C07D 471/18; A61K 31/55; C07D 491/22

(52) U.S. Cl. CPC C07D 471/18; A61K 31/55; C07D 491/22

(57) ABSTRACT

Provided herein are ibogaine compounds which can be useful for methods of treating a neuropsychiatric disease or neurological disorder, for increasing neural plasticity, or for modulating the function of a serotonin transporter.

### CROSS-REFERENCE TO RELATED APPLICATIONS

This application claims priority to U.S. Provisional Application No. 63/564,092, filed Mar. 12, 2024, which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under GM148182, awarded by the National Institutes of Health (NIH). The Government has certain rights in this invention.

### BACKGROUND

Altered synaptic connectivity and plasticity has been observed in the brains of individuals with neuropsychiatric and neurological diseases/disorders. Psychoplastogens promote neuronal growth and improve neuronal architecture through a variety of mechanisms. Modulators of these biological targets, such as, for example, N,N-dimethyltryptamine (DMT), ibogaine, and lysergic acid diethylamide (LSD) have demonstrated psychoplastogenic properties. For example, LSD and other analogs of the ergoline scaffold are capable of rectifying deleterious changes in neuronal structure that are associated with neuropsychiatric and neurological diseases/disorders. Such structural alterations include, for example, the loss of dendritic spines and synapses in the prefrontal cortex (PFC) as well as reductions in dendritic arbor complexity. Furthermore, pyramidal neurons in the PFC exhibit top-down control over areas of the brain controlling motivation, fear, reward, and cognition. Hallucinogenic psychoplastogens have demonstrated antidepressant, anxiolytic, and anti-addictive effects in the clinic. What is needed are new compounds that promote cortical neuron growth and neuroplasticity.

### BRIEF SUMMARY OF THE INVENTION

In one embodiment, provided herein is a compound, or a pharmaceutically acceptable salt thereof, having a structure of Formula I:

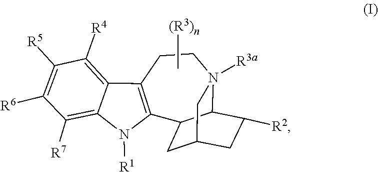

wherein:
R¹ is hydrogen or C₁₋₆ alkyl;
R² is hydrogen or C₁₋₆ alkyl;
each R³ is independently hydrogen, C₁₋₆ alkyl, C₃₋₈ cycloalkyl, or C₄₋₁₄ alkyl-cycloalkyl;
R^{3a} is absent, hydrogen or C₁₋₆ alkyl;
R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ hydroxyalkyl, C₂₋₆ alkoxyalkyl, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl, wherein at least one of R⁴, R⁵, R⁶ and R⁷ is not H;
alternatively, R⁴ and R⁵, R⁵ and R⁶, or R⁶ and R⁷ are combined with the atoms to which they are each attached to form a C₃₋₆ cycloalkyl, heterocycloalkyl, C₆₋₁₂ aryl or heteroaryl;
R^{8a}, R^{8b}, R^{8c} and R^{8d} are each independently H, C₁₋₆ alkyl; and
subscript n is 0, 1, 2, or 3;
wherein each heterocycloalkyl has 3 to 10 ring members and 1 to 4 heteroatoms each independently N, O, or S, and each heteroaryl has 5 to 10 ring members and 1 to 4 heteroatoms each independently N, O, or S,
wherein when R² is ethyl, then R⁵ is F and/or R⁷ is —OMe, and
wherein when R² is H, then R⁵ is other than H and —OMe.

In another embodiment, provided herein is a pharmaceutical composition, comprising a therapeutically effective amount of a compound of the present invention, or a pharmaceutically acceptable salt thereof.

In another embodiment, provided herein is a method of treating a disease, comprising administering to a subject in need thereof, a therapeutically effective amount of a compound of the present invention, or a pharmaceutically acceptable salt thereof, thereby treating the disease.

In another embodiment, provided herein is a method for increasing neural plasticity, the method comprising contacting a neuronal cell with a compound of the present invention, or a pharmaceutically acceptable salt thereof, in an amount sufficient to increase neural plasticity of the neuronal cell.

In another embodiment, provided herein is a method for increasing neural plasticity and increasing dendritic spine density, the method comprising contacting a neuronal cell with a compound of the present invention, or a pharmaceutically acceptable salt thereof, in an amount sufficient to increase neural plasticity and increase dendritic spine density of the neuronal cell.

In another embodiment, provided herein is a method for modulating the function of a serotonin transporter, the method comprising contacting the serotonin transporter with a compound of the present invention, or a pharmaceutically acceptable salt thereof, in an amount sufficient to modulate the function of the serotonin transporter.

### BRIEF DESCRIPTION OF THE DRAWINGS

FIG. 1A shows the completed synthesis of epi ibogaine. Step-count is indicated by the numbers over each reaction arrow. Calculations of thermodynamic stability indicated that isomer 10 was lower in energy than isomer 11. FIG. 1B shows that HAT reduction of 13a only yielded the endo product 17a. Calculations of thermodynamic stability indicated that the C20 exo isomer 14b was lower in energy than endo isomer 14a. FIG. 1C shows that carbamate coordination of a metallo radical intermediate could potentially explain the selectivity observed in HAT reactions.

FIG. 2A shows the completed synthesis of (±)-ibogaine. Step-count is indicated by the numbers over each reaction arrow. FIG. 2B shows that ¹H NMR data demonstrates that synthetic and natural ibogaine are indistinguishable. FIG. 2C shows that synthetic efficiency was assessed by comparing overall yield, total step count, and quantity of ibogaine produced following the final step (i.e., scale) for the 4 reported total syntheses of (±)-ibogaine (left). The modularity of the synthesis was evaluated by plotting Bottcher's complexity index vs. step count (i.e., percent through the synthesis) (right). FIG. 2D shows structures of additional natural products that were synthesized using our synthetic strategy. Overall yields for their syntheses are highlighted in blue. Compounds with a hydrogen or ethyl group at C20 were synthesized in 6 or 7 steps overall, respectively. FIG. 2E shows structures of non-natural analogues that were synthesized using our synthetic strategy. Overall yields for their syntheses are highlighted in blue. Compounds with a hydrogen or ethyl group at C20 were synthesized in 6 or 7 steps overall, respectively.

FIG. 3A shows that dendritic spine density on rat embryonic cortical neurons (DIV19) was assessed after treatment (10 μM) for 24 h. Neurons were visualized using a fluorescent conjugate of phalloidin. Unlike natural (−)-ibogaine, non-natural (+)-ibogaine does not promote spine growth. In contrast. (−)-29 promotes spine growth comparable to (−)-ibogaine. (N=17 neurons for VEH treatment, N=18 neurons for all other treatments); one-way ANOVA with Dunnett's post hoc test). Data are presented as mean±SEM. *P<0.05, **P<0.01, ***P<0.001, ****P<0.0001, as compared to the VEH control (VEH vs. BDNF, P=0.0472; VEH vs. (+)-IBO, P=0.9760; VEH vs. (−)-IBO, P=<0.0001; VEH vs. (−)-F-IBO, P=0.0016; VEH vs. nor-IBO, P=<0.0001). FIG. 3B shows representative images of dendritic branches following treatment. Scale bar=5 mm. FIG. 3C shows that noribogaine and (−)-29 promote efflux of accumulated [³H]5-HT from HEK293T cells heterologously expressing SERT. Compounds were treated at 10 μM. (N=7-11 wells per treatment from 3 independent cultures; one-way ANOVA with Dunnett's post hoc test). *P<0.05, **P<0.01, ***P<0.001, ****P<0.0001, as compared to the VEH control. VEH=vehicle; BDNF=brain-derived neurotrophic factor; (−)-IBO=(−)-ibogaine 1; (+)-IBO=(+)-ibogaine; nor-IBO=noribogaine; (−)-F-IBO=(−)-10-fluoroibogamine 29; EC=radioactivity in the extracellular sample; IC=radioactivity in the intracellular sample.

FIG. 4A shows structures of major iboga alkaloids. FIG. 4B shows common synthetic strategies for accessing the iboga core scaffold. The order of indole (A ring), tetrahydroazepine (B ring) and isoquinuclidine (C ring) construction is indicated. FIG. 4C shows that our retrosynthetic analysis suggested that constructing the indole (A ring) last would enable access to multiple natural products and analogues.

FIG. 5A shows the completed asymmetric total synthesis of (+)-ibogaine. Step-count is indicated by the numbers over each reaction arrow. FIG. 5B shows structures of additional non-natural ibogaine analogues that were synthesized using our asymmetric synthetic strategy, (+)-ibogaine, (−)-10-fluoroibogamine, and (+)-10-fluoroibogamine. Overall yields for their syntheses are highlighted in blue. PMPH=4-methoxy phenylhydrazine.

FIG. 6A shows further biological effects of iboga alkaloids and related analogues and that both (+)-29 and (−)-29 inhibit the uptake of [³H]5-HT into HEK293T cells heterologously expressing SERT. Data were normalized to the vehicle control (0%) and 100 μM cocaine (100%). FIG. 6B shows further biological effects of iboga alkaloids and related analogues and that noribogaine and (−)-29 promote partial efflux of accumulated [³H]5-HT from HEK293T cells heterologously expressing SERT. Data were normalized to the vehicle control (0) %) and 100 μM PCA (100%) (N=3 wells per treatment from 2 independent cultures, and data are presented as mean±SEM). VEH=vehicle; BDNF=brain-derived neurotrophic factor; (−)-IBO=(−)-ibogaine 1; (+)-IBO=(+)-ibogaine; nor-IBO=noribogaine; (−)-F-IBO=(−)-10-fluoroibogamine 29; (+)-F-IBO=(+)-10-fluoroibogamine 29, 5-HT=serotonin, PCA=p-chloroamphetamine, Coca=cocaine, (±)-MDMA=(±)-3,4-methylenedioxy methamphetamine.

FIG. 7A shows further biological effects of iboga alkaloids and related analogues and that Compound 29 promotes partial efflux of radiolabeled serotonin ([³H]5-HT) from HEK293T cells expressing SERT. FIG. 7B shows further biological effects of iboga alkaloids and related analogues and that Compound 31 promotes partial efflux of radiolabeled serotonin ([³H]5-HT) from HEK293T cells expressing SERT.

FIG. 8A shows cyclopropyl peak correlations (CH₂ and CH) through homonuclear correlation spectroscopy (COSY) spectra for the endo and exo epimers of intermediate 13 (intermediates 13a and 13b). FIG. 8B shows the ¹H NMR spectra for the endo and exo forms of intermediate 13 (intermediates 13a and 13b) and 1D selective gradient NOESY spectra. Observed correlations through ID selective NOESY shows correlations between methine of cyclopropyl in endo epimer, but not in exo epimer.

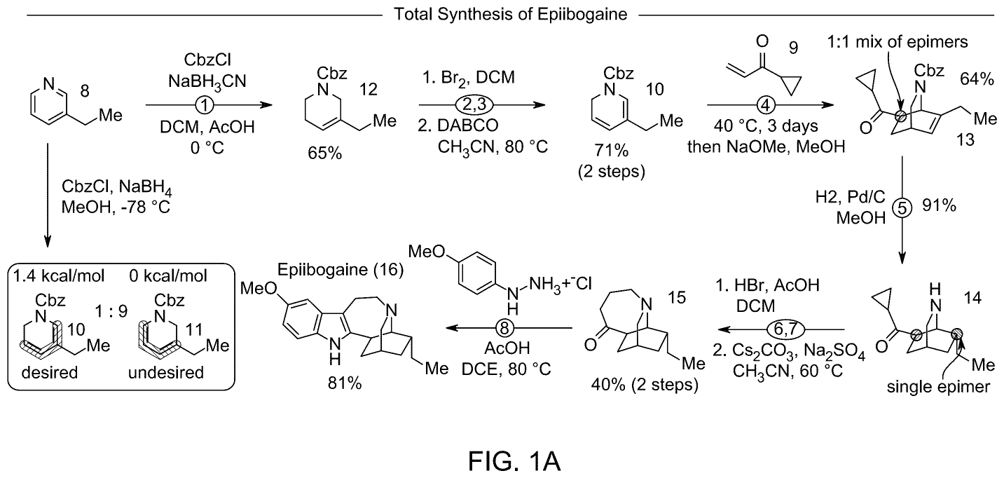

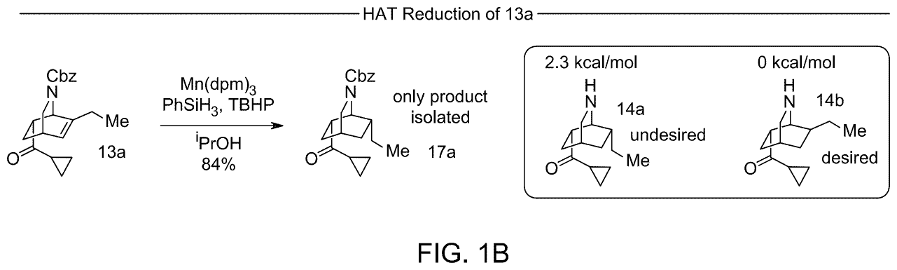

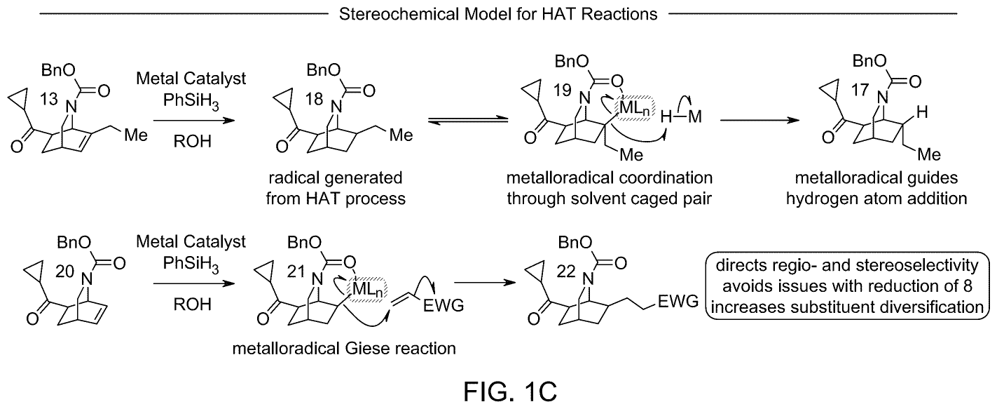

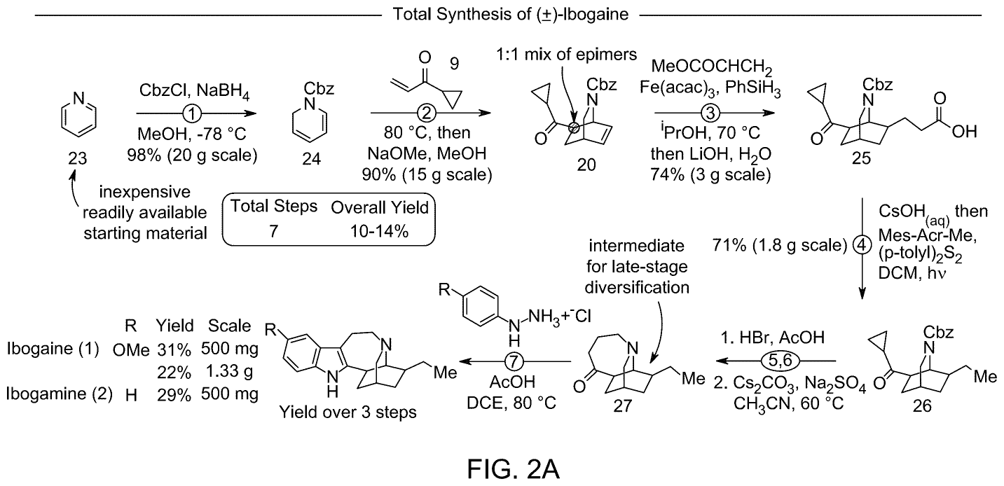

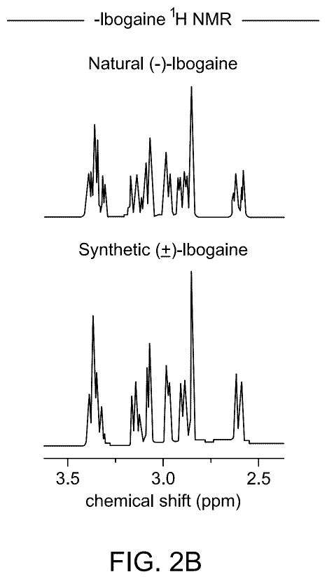

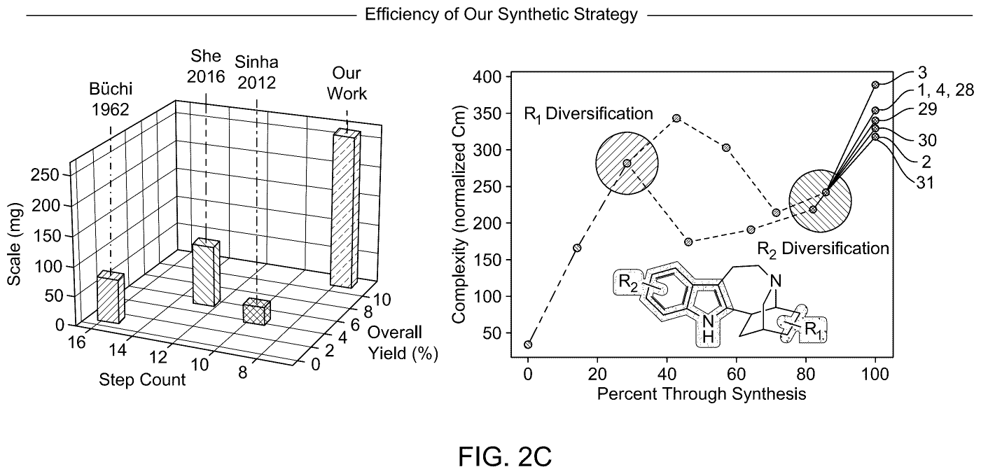

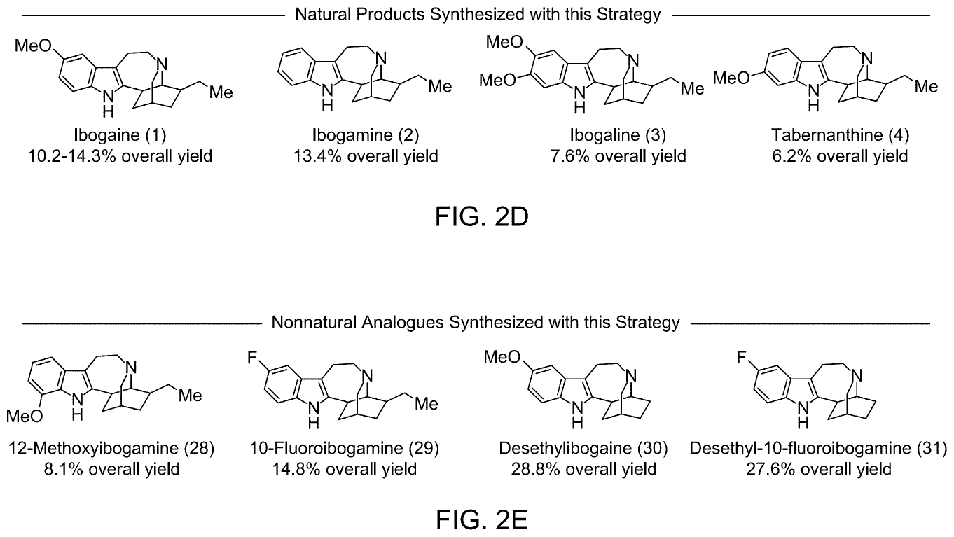

### DETAILED DESCRIPTION OF THE INVENTION

### I. General

Provided herein are ibogaine compounds. The compounds of the present invention are useful for treatment of diseases, such as brain disorders, neuropsychiatric diseases, and other neurological diseases. The compounds of the present invention are also useful for increasing neural plasticity, increasing dendritic spine density, or both.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Disclosures provided herein of an “alkyl” are intended to include independent recitations of a saturated alkyl, unless otherwise stated. Alkyl groups described herein are generally monovalent, but may also be divalent which may also be described herein as “alkylene” or “alkylenyl” groups. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatrienyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The alkoxy component is as defined above. Examples of the alkoxyalkyl group include, but are not limited to, 2-ethoxy-ethyl and methoxymethyl.

“Alkylhydroxyl” or “hydroxyalkyl” refers to an alkyl group, as defined above, where at least one of the hydrogen atoms is replaced with a hydroxy group. As for the alkyl group, alkylhydroxyl groups can have any suitable number of carbon atoms, such as C₁₋₆. Exemplary alkylhydroxyl groups include, but are not limited to, hydroxy-methyl, hydroxyethyl (where the hydroxy is in the 1- or 2-position), hydroxypropyl (where the hydroxy is in the 1-, 2- or 3-position), hydroxy butyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy. 2,2,2-trifluoroethoxy, perfluoroethoxy, etc.

“Alkylamine” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. In some embodiments, cycloalkyls are spirocyclic or bridged compounds. In some embodiments, cycloalkyls are optionally fused with an aromatic ring, and the point of attachment is at a carbon that is not an aromatic ring carbon atom. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane. [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Cycloalkyl groups can contain one or more double bonds in the ring.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” refers to a saturated ring system having from 3 to 12 ring members and from 1 to 4 heteroatoms of N, O and S. The heteroatoms can also be oxidized, such as, but not limited to, —S(O)-and-S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. In some embodiments, heterocycloalkyls are spirocyclic or bridged compounds. In some embodiments, heterocycloalkyls are optionally fused with an aromatic ring, and the point of attachment is at a carbon or heteroatom (e.g., nitrogen atom) that is not an aromatic ring carbon atom. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperadinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{y}—OR^{x}, —R^{y}—OC(O)—R^{x}, —R^{y}—OC(O)—OR^{x}—R^{y}—OC(O)—N(R^{x})₂, —R^{y}—N(R^{x})₂, —R^{y}—C(O)R^{x}, —R^{y}—C(O)OR^{x}—R^{y}—C(O)N(R^{x})₂, —R^{y}—O—R^{z}—C(O)N(R^{x})₂, —R^{y}—N(R^{x})C(O) OR^{x}, —R^{y}—N(R^{x})C(O)R^{x}, —R^{y}—N(R^{x})S(O)ₜR^{x} (where t is 1 or 2), —R^{y}—S(O)ₜR^{x} (where t is 1 or 2), —R^{y}—S(O)ₜOR^{x} (where t is 1 or 2) and —R^{y}—S(O)ₜN(R^{x})₂ (where t is 1 or 2), where each R^{x} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{y} is independently a direct bond or a straight or branched alkylene or alkenylene chain, and R^{z} is a straight or branched alkylene or alkenylene chain, and where each of the above substituents is unsubstituted unless otherwise indicated.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above. Alkyl-heterocycloalkyl groups can be substituted or unsubstituted.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxy methyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter: including, e.g., the result of a physical examination.

“Disease” refers abnormal cellular function in an organism, which is not due to a direct result of a physical or external injury. Diseases can refer to any condition that causes distress, dysfunction, disabilities, disorders, infections, pain, or even death. Diseases include, but are not limited to hereditary diseases such as genetic and non-genetic diseases, infectious diseases, non-infectious diseases such as cancer, deficiency diseases, neurological diseases, and physiological diseases.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Neural plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Dendritic crossing” refers to dendritic branches which overlap each other or form a cluster. Dendritic crossing can be measured by Sholl Analysis.

“Dendritic spine” refers to the small membrane protruding from a dendrite which can receive electric signal from an axon at the synapse. Dendritic spines are useful for transmitting electric signals to the neuron's cell body. Dendrites of a single neuron can comprise hundreds to thousands of spines. Dendritic spine density refers to the number of spines within the length of a dendrite. As an illustrative example, a dendritic spine density of 5 μm⁻¹ indicates 5 spines per 1 μm stretch of a dendrite.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A} or 5HT_{2C}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 μM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist. “Functionally selective agonist” refers to a modulator that produces one or a subset of biological responses that are possible from activation of a receptor. For example, activation of 5HT_{2A} receptors is known to cause many downstream effects including increased neural plasticity, increased intracellular calcium concentrations, and hallucinations, among many other biological responses. A functionally selective agonist would produce only a subset of the biological responses possible from activation of the 5HT_{2A} receptor.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur. “Functionally selective antagonists” block one signaling pathway while leaving others in tact.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“Modulate a serotonin transporter” refers to any compound which modulates any biological response associated with the binding of a serotonin transporter to an agonist.

### III. Compounds

The present invention provides ibogaine compounds of Formula I, Ia, and Ib, useful for the treatment of a variety of neurological diseases and disorders as well as increasing neuronal plasticity.

In some embodiments, provided herein is a compound, or a pharmaceutically acceptable salt thereof, having a structure of Formula I:

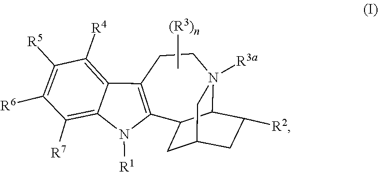

wherein:
R¹ is hydrogen or C₁₋₆ alkyl;
R² is hydrogen or C₁₋₆ alkyl;
each R³ is independently hydrogen, C₁₋₆ alkyl, C₃₋₈ cycloalkyl, or C₄₋₁₄ alkyl-cycloalkyl;
R^{3a} is absent, hydrogen or C₁₋₆ alkyl;
R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ hydroxyalkyl, C₂₋₆ alkoxyalkyl, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}, R^{8d}), —C(O)C(O)N(R^{8b}R^{8c}), —S(O₂)R^{8b}, —S(O)₂N(R^{8b}, R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl, wherein at least one of R⁴, R⁵, R⁶ and R⁷ is not H;
alternatively, R⁴ and R⁵, R⁵ and R⁶, or R⁶ and R⁷ are combined with the atoms to which they are each attached to form a C₃₋₆ cycloalkyl, heterocycloalkyl, C₆₋₁₂ aryl or heteroaryl;
R^{8a}, R^{8b}, R^{8c} and R^{8d} are each independently H, C₁₋₆ alkyl; and subscript n is 0, 1, 2, or 3;
wherein each heterocycloalkyl has 3 to 10 ring members and 1 to 4 heteroatoms each independently N, O, or S, and each heteroaryl has 5 to 10 ring members and 1 to 4 heteroatoms each independently N, O, or S,
wherein when R² is ethyl, then R⁵ is F and/or R⁷ is —OMe, and
wherein when R² is H, then R⁵ is other than H and —OMe.

In some embodiments, the compound of Formula I, or a pharmaceutically acceptable salt thereof, is the compound wherein R¹ is hydrogen.

In some embodiments, the compound of Formula I, or a pharmaceutically acceptable salt thereof, is the compound wherein the compound has the structure of Formula (Ia):

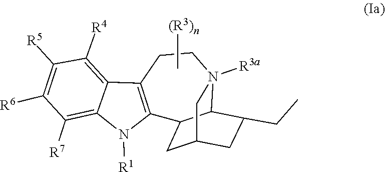

wherein R⁵ is F and/or R⁷ is —OMe.

In some embodiments, the compound of Formula Ia, or a pharmaceutically acceptable salt thereof, is the compound wherein
R⁴, R⁵, R⁶ and R⁷ are each independently hydrogen, C₁₋₆ alkyl, halogen, C₁₋₆ haloalkyl, C₁₋₆ alkoxy, C₂₋₆ alkoxyalkyl, or C₁₋₆ haloalkoxy; and
alternatively, R⁴ and R⁵, R⁵ and R⁶, or R⁶ and R⁷ are combined with the atoms to which they are each attached to form a heterocycloalkyl having 5 to 6 ring members and 1 or 2 heteroatoms each independently O or S,
wherein at least one of R⁴, R⁵, R⁶ and R⁷ is not H, and
wherein R⁵ is F and/or R⁷ is —OMe.

In some embodiments, the compound of Formula Ia, or a pharmaceutically acceptable salt thereof, is the compound wherein
R⁴, R⁵, R⁶ and R⁷ are each independently hydrogen, methyl, ethyl, iso-propyl, F, Cl, Br, —CF₃, —OMe, OEt, -OiPr, —CH₂OMe, or —OCF₃; and
alternatively, R⁴ and R⁵, R⁵ and R⁶, or R⁶ and R⁷ are combined with the atoms to which they are each attached to form a heterocycloalkyl having 5 ring members and 2 heteroatoms each independently O or S,
wherein at least one of R⁴, R⁵, R⁶ and R⁷ is not H, and
wherein R⁵ is F and/or R⁷ is —OMe.

In some embodiments, the compound of Formula Ia, or a pharmaceutically acceptable salt thereof, is the compound wherein
R⁴, R⁵, R⁶ and R⁷ are each independently hydrogen, F, Cl, or —OMe; and
alternatively, R⁴ and R⁵, R⁵ and R⁶, or R⁶ and R⁷ are combined with the atoms to which they are each attached to form a heterocycloalkyl having 5 ring members and 2 heteroatoms each O,
wherein at least one of R⁴, R⁵, R⁶ and R⁷ is not H, and
wherein R⁵ is F and/or R⁷ is —OMe.

In some embodiments, the compound of Formula I or Ia, or a pharmaceutically acceptable salt thereof, is the compound having the structure:

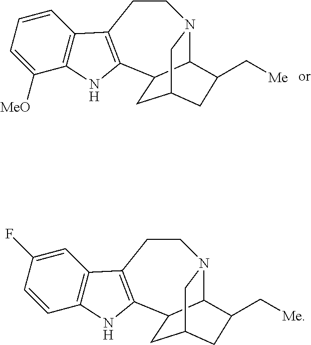 [SMILES: CCC1CC2CC3c4[nH]c5c(OC)cccc5c4CCN(C2)C13 ; CCC1CC2CC3c4[nH]c5ccc(F)cc5c4CCN(C2)C13]

In some embodiments, the compound of Formula I, or a pharmaceutically acceptable salt thereof, is the compound having a structure of Formula (Ib):

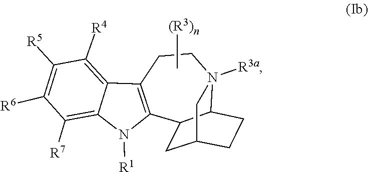

wherein
R¹ is hydrogen or C₁₋₆ alkyl;
each R³ is independently hydrogen, C₁₋₆ alkyl, C₃₋₈ cycloalkyl, or C₄₋₁₄ alkyl-cycloalkyl;
R^{3a} is absent, hydrogen or C₁₋₆ alkyl;
R⁴, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ hydroxyalkyl, C₂₋₆ alkoxyalkyl, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl;
R⁵ is C₁₋₆ alkyl, C₂₋₆ alkenyl, C₂₋₆ alkynyl, halogen, C₁₋₆ haloalkyl, C₁₋₆ alkylamine, C₂₋₆ alkoxy, C₁₋₆ hydroxyalkyl, C₂₋₆ alkoxyalkyl, C₁₋₆ haloalkoxy, —OH, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl;
alternatively, R⁴ and R⁵, R⁵ and R⁶, or R⁶ and R⁷ are combined with the atoms to which they are each attached to form a C₃₋₆ cycloalkyl, heterocycloalkyl, C₆₋₁₂ aryl or heteroaryl;
R^{8a}, R^{8b}, R^{8c} and R^{8d} are each independently H, C₁₋₆ alkyl; and
subscript n is 0, 1, 2, or 3,
wherein each heterocycloalkyl has 3 to 10 ring members and 1 to 4 heteroatoms each independently N, O, or S, and each heteroaryl has 5 to 10 ring members and 1 to 4 heteroatoms each independently N, O, or S

In some embodiments, the compound of Formula I or Ib, or a pharmaceutically acceptable salt thereof, is the compound wherein subscript n is 0.

In some embodiments, the compound of Formula Ib, or a pharmaceutically acceptable salt thereof, is the compound wherein
R⁴, R⁶ and R⁷ are each independently hydrogen, C₁₋₆ alkyl, halogen, C₁₋₆ haloalkyl, C₁₋₆ alkoxy, C₂₋₆ alkoxyalkyl, or C₁₋₆ haloalkoxy; and
R⁵ is C₁₋₆ alkyl, halogen, C₁₋₆ haloalkyl, C₂₋₆ alkoxy, C₂₋₆ alkoxyalkyl, or C₁₋₆ haloalkoxy;
alternatively, R⁴ and R⁵, R⁵ and R⁶, or R⁶ and R⁷ are combined with the atoms to which they are each attached to form a heterocycloalkyl having 5 to 6 ring members and 1 or 2 heteroatoms each independently O or S.

In some embodiments, the compound of Formula Ib, or a pharmaceutically acceptable salt thereof, is the compound wherein
R⁴, R⁶ and R⁷ are each independently hydrogen, methyl, ethyl, iso-propyl, F, Cl, Br, —CF₃, —OMe, OEt, -OiPr, —CH₂OMe, or —OCF₃; and
R⁵ is methyl, ethyl, iso-propyl, F, Cl, Br, —CF₃, OEt, -OiPr, —CH₂OMe, or —OCF₃;
alternatively, R⁴ and R⁵, R⁵ and R⁶, or R⁶ and R⁷ are combined with the atoms to which they are each attached to form a heterocycloalkyl having 5 ring members and 2 heteroatoms each independently O or S.

In some embodiments, the compound of Formula Ib, or a pharmaceutically acceptable salt thereof, is the compound wherein
R⁴, R⁶ and R⁷ are each independently hydrogen, F, Cl, or —OMe; and
R⁵ is F, or Cl;
alternatively, R⁴ and R⁵, R⁵ and R⁶, or R⁶ and R⁷ are combined with the atoms to which they are each attached to form a heterocycloalkyl having 5 ring members and 2 heteroatoms each O.

In some embodiments, the compound of Formula Ib, or a pharmaceutically acceptable salt thereof, is the compound having the structure:

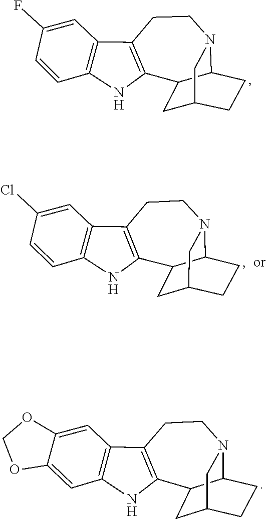 [SMILES: Fc1ccc2[nH]c3c(c2c1)CCN1CC2CCC1C3C2 ; Clc1ccc2[nH]c3c(c2c1)CCN1CC2CCC1C3C2 ; c1c2c(cc3c4c([nH]c13)C1CC3CCC1N(CC4)C3)OCO2]

In some embodiments, the compound of Formula Ib, or a pharmaceutically acceptable salt thereof, is the compound having the structure:

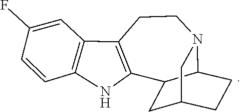 [SMILES: Fc1ccc2[nH]c3c(c2c1)CCN1CC2CCC1C3C2]

The compounds of the present invention can also be in the salt forms, such as acid or base salts of the compounds of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The present invention also includes isotopically-labeled compounds of the present invention, wherein one or more atoms are replaced by one or more atoms having specific atomic mass or mass numbers. Examples of isotopes that can be incorporated into compounds of the invention include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). Isotopically-labeled compounds of the present invention are useful in assays of the tissue distribution of the compounds and their prodrugs and metabolites; preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of this invention can generally be prepared according to the methods known by one of skill in the art by substituting an isotopically-labeled reagent for a non-isotopically labeled reagent. Compounds of the present invention can be isotopically labeled at positions adjacent to the basic amine, in aromatic rings, and the methyl groups of methoxy substituents.

The present invention includes all tautomers and stereoisomers of compounds of the present invention, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. Compounds according to the present invention can be prepared using diastereomers, enantiomers or racemic mixtures as starting materials. Furthermore, diastereomer and enantiomer products can be separated by chromatography, fractional crystallization or other methods known to those of skill in the art.

In some embodiments, a compound provided herein, including pharmaceutically acceptable salts and solvates thereof, is a non-hallucinogenic psychoplastogen. In some embodiments, the non-hallucinogenic psychoplastogen promotes neuronal growth, improves neuronal structure, or a combination thereof.

### IV. Pharmaceutical Compositions and Formulations

In some embodiments, provided herein is a pharmaceutical composition, comprising a therapeutically effective amount of a compound of the present invention, or a pharmaceutically acceptable salt thereof.

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co. Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxy propylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxy benzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg. 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### V. Methods of Treatment

In some embodiments, provided herein is a method of treating a disease or disorder, such as, but not limited to a neurological disease or disorder, comprising administering to a subject in need thereof, a therapeutically effective amount of a compound of the present invention, or a pharmaceutically acceptable salt thereof, thereby treating the disease or disorder.

In some embodiments, provided herein is a method of treating a disease, comprising administering to a subject in need thereof, a therapeutically effective amount of a compound of the present invention, or a pharmaceutically acceptable salt thereof, thereby treating the disease.

### Neurological Disorders

Neuronal plasticity, and changes thereof, have been attributed to many neurological diseases and disorders. For example, during development and in adulthood, changes in dendritic spine number and morphology (e.g., lengths, crossings, density) accompany synapse formation, maintenance and elimination: these changes are thought to establish and remodel connectivity within neuronal circuits. Furthermore, dendritic spine structural plasticity is coordinated with synaptic function and plasticity. For example, spine enlargement is coordinated with long-term potentiation in neuronal circuits, whereas long-term depression is associated with spine shrinkage.

In addition, dendritic spines undergo experience-dependent morphological changes in live animals, and even subtle changes in dendritic spines can affect synaptic function, synaptic plasticity, and patterns of connectivity in neuronal circuits. For example, disease-specific disruptions in dendritic spine shape, size, and/or number accompany neurological diseases and disorders, such as, for example, neurodegenerative (e.g., Alzheimer's disease or Parkinson's disease) and neuropsychiatric (e.g., depression or schizophrenia) diseases and disorders, suggesting that dendritic spines may serve as a common substrate in diseases that involve deficits in information processing.

Unless indicated otherwise, a neurological disease or disorder generally refers to a disease or disorder of the central nervous system (CNS) (e.g., brain, spine, and/or nerves) of an individual.

In some embodiments, provided herein is a method of treating a neurological disease or disorder with a compound provided herein (e.g., a compound of Formula (I), Formula (Ia), Formula (Ib), or a pharmaceutically acceptable salt or solvate thereof).

In some embodiments, a compound provided herein, or a pharmaceutically acceptable salt or solvate thereof) improves dendritic spine number and dendritic spine morphology that is lost in a neurological disease or disorder.

In some embodiments, a compound of the present invention is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the disease is headache disorders. In some embodiments, the neurological disease is a migraine or cluster headache. In some embodiments, the disease is migraines. In some embodiments, the disease is cluster headaches. In some embodiments, the disease is addiction. In some embodiments, the disease is substance use disorder. In some embodiments, the disease is alcohol use disorder. In some embodiments, the disease is alcohol use disorder.

In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is an anxiety disorder. In some embodiments, the neuropsychiatric disease or neurological disease is anxiety. In some embodiments, the neuropsychiatric disease or neurological disease is post-traumatic stress disorder (PTSD). In some embodiments, the neurological disease is autism. In some embodiments, the neurological disease is stroke or traumatic brain injury. In some embodiments, the neuropsychiatric disease or neurological disease is schizophrenia.

In some embodiments, the disease is a neuropsychiatric disease. In some embodiments, the diseases is a neurodegenerative disease.

In some embodiments, a compound of the present invention is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, schizophrenia, and addiction (e.g., substance abuse disorder). In some embodiments, brain disorders include, for example, migraines, addiction (e.g., substance use disorder), depression, and anxiety.

In some embodiments, provided herein is a method for increasing neural plasticity, the method comprising contacting a neuronal cell with a compound of the present invention, or a pharmaceutically acceptable salt thereof, in an amount sufficient to increase neural plasticity of the neuronal cell.

The neuronal plasticity can be measured by a variety of methods, including but not limited to, the average number of branch points per neuron, the average total neurite length per neuron, and the average number of neurites per neuron. For example, the average number of branch points per neuron can be at least 1.0, 1.05, 1.1, 1.15, 1.2, or at least 1.25. Alternatively, the branch points per neuron can be at least 1.05 fold above the vehicle control, or at least 1.1, 1.15, 1.2, or at least 1.25 fold above the vehicle control. The average total neurite length per neuron can be at least 100 μm, or at least 105, 110, 115, 120, 125, 130, 135, 140, 145, or at least 150 μm. Alternatively, the neurite length per neuron can be at least 1.05 fold above the vehicle control, or at least 1.1, 1.15, 1.2, or at least 1.25 fold above the vehicle control. The average number of neurites per neuron of can be at least 2.8, or at least 2.9, 3.0, 3.1, 3.2, 3.3, 3.4, or at least 3.5. Alternatively, the neurites per neuron can be at least 1.05 fold above the vehicle control, or at least 1.1, 1.15, 1.2, or at least 1.25 fold above the vehicle control.

Neural plasticity refers to the ability of the brain to change structure and/or function throughout a subject's life. New neurons can be produced and integrated into the central nervous system throughout the subject's life. Increasing neural plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neural plasticity comprises promoting neuronal growth, promoting neuritogenesis, promoting synaptogenesis, promoting dendritogenesis, increasing dendritic arbor complexity, and increasing dendritic spine density.

In some embodiments, increasing neural plasticity can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder. In some embodiments, the neuropsychiatric disease is bipolar disorder. In some embodiments, the disease is depression. In some embodiments, the disease is a neurodegenerative disease. In some embodiments, the disease is Alzheimer's disease or Parkinson's disease. In some embodiments, the disease is Alzheimer's disease. In some embodiments, the disease is Parkinson's disease.

In some embodiments, a compound of the present invention is used to increase neural plasticity. In some embodiments, the compounds used to increase neural plasticity have, for example, anti-addictive properties, antidepressant properties, anxiolytic properties, or a combination thereof. In some embodiments, decreased neural plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety. In some embodiments, the disease is a neuropsychiatric disease.

In some embodiments, the experiment or assay to determine increased neural plasticity of any compound of the present invention is a phenotypic assay, a dendritogenesis assay, a spinogenesis assay, a synaptogenesis assay, a Sholl analysis, or a concentration-response experiment. In some embodiments, the experiment or assay to determine the hallucinogenic potential of any compounds of the present invention is a mouse head-twitch response (HTR) assay.

In some embodiments, provided herein is a method for increasing neural plasticity and increasing dendritic spine density, the method comprising contacting a neuronal cell with a compound of the present invention, or a pharmaceutically acceptable salt thereof, in an amount sufficient to increase neural plasticity and increase dendritic spine density of the neuronal cell.

Dendritic spines are dynamic and can have significant changes in density, shape, and volume over time. The growth or loss of dendritic spines, which contribute to the dendritic spine density, can be important for reinforcing neural pathways for learning, memory, and general cognitive function. Increasing dendritic spine density can be useful for treatment of neurological diseases, such as, but not limited to, neurodegenerative diseases and neuropsychiatric diseases.

Increasing dendritic spine density can be measured by staining and immunocytochemical methods known by one of skill in the art. Staining methods include, but are not limited to electron microscopy, Golgi staining, crystal violet staining, DAPI staining, and eosin staining. For example, Golgi staining can be used to measure dendritic spine density.

In some embodiments, a compound provided herein, or pharmaceutically acceptable salts thereof, is useful for promoting neuronal growth and/or improving neuronal structure.

In some embodiments, a compound provided herein, or pharmaceutically acceptable salts thereof, is a non-hallucinogenic psychoplastogens useful for treating one or more diseases or disorders associated with loss of synaptic connectivity and/or plasticity.

In some embodiments, an individual administered a compound provided herein does not have a hallucinogenic event (e.g., at any point after the compound has been administered to the individual).

In some embodiments, provided herein is a method for treating a disease or disorder in an individual in need thereof, wherein the disease or disorder is a neurological diseases and disorder.

In some embodiments, a compound provided herein, or a pharmaceutically acceptable salt thereof, is used in the preparation of medicaments for the treatment of diseases or conditions in a mammal that would benefit from promoting neuronal growth and/or improving neuronal structure.

Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a mammal already suffering from a disease or condition, in an amount sufficient to cure or at least partially arrest at least one of the symptoms of the disease or condition. Amounts effective for this use depend on the severity and course of the disease or condition, previous therapy, the mammal's health status, weight, and response to the drugs, and the judgment of a healthcare practitioner. Therapeutically effective amounts are optionally determined by methods including, but not limited to, a dose escalation and/or dose ranging clinical trial.

In prophylactic applications, compositions containing the compounds described herein are administered to a mammal susceptible to or otherwise at risk of a particular disease, disorder or condition. Such an amount is defined to be a “prophylactically effective amount or dose.” In this use, the precise amounts also depend on the mammal's state of health, weight, and the like. When used in mammals, effective amounts for this use will depend on the severity and course of the disease, disorder or condition, previous therapy, the mammal's health status and response to the drugs, and the judgment of a healthcare professional. In some embodiments, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In some embodiments wherein the mammal's condition does not improve, upon the discretion of a healthcare professional the administration of the compounds are administered chronically, that is, for an extended period of time, including throughout the duration of the mammal's life in order to ameliorate or otherwise control or limit the symptoms of the mammal's disease or condition.

In some embodiments wherein a mammal's status does improve, the dose of drug being administered is temporarily reduced or temporarily suspended for a certain length of time (i.e., a “drug holiday”). In some embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In some embodiments, however, the mammal requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In some embodiments, doses employed for adult human treatment are from about 1 mg to about 1000 mg per day. In some embodiments, the desired dose is conveniently presented in a single dose or in divided doses administered simultaneously or at appropriate intervals, for example as two, three, four or more sub-doses per day.

In some embodiments, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In some embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In some embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In some embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In some embodiments, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In some embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the disease(s) for which relief is sought, is modified in accordance with a variety of factors (e.g. the disease or disorder from which the subject suffers: the age, weight, sex, diet, and medical condition of the subject). Thus, in some instances, the dosage regimen actually employed varies and, in some embodiments, deviates from the dosage regimens set forth herein.

In some embodiments, provided herein is a method for modulating the function of a serotonin transporter, the method comprising contacting the serotonin transporter with a compound of the present invention, or a pharmaceutically acceptable salt thereof, in an amount sufficient to modulate the function of the serotonin transporter. In some embodiments, modulating the serotonin transporter includes one or more of inhibiting serotonin uptake and facilitating serotonin efflux.

### VI. EXAMPLES

### A. General Methods

All reagents were obtained from commercial sources and reactions were performed using oven-dried glassware (120° C.) under an inert N₂ atmosphere unless otherwise noted. Air- and moisture-sensitive liquids and solutions were transferred via syringe or stainless-steel cannula. Organic solutions were concentrated under reduced pressure (˜5 Torr) by rotary evaporation. Solvents were purified by passage under 12 psi N₂ through activated alumina columns. Chromatography was performed using Fisher Chemical™ Silica Gel Sorbent (230-400 Mesh, Grade 60). Compounds purified by chromatography were typically applied to the adsorbent bed using the indicated solvent conditions with a minimum amount of added chloroform as needed for solubility. Thin layer chromatography (TLC) was performed on Merck silica gel 60 F254 plates (250 μm). Visualization of the developed chromatogram was accomplished by fluorescence quenching or by staining with iodine, butanoic ninhydrin, aqueous potassium permanganate, or aqueous ceric ammonium molybdate (CAM). Irradiation of photochemical reactions was carried out using 2 HIGROW LED Aquarium Light Blub, Wolezek 30 W LED Plant Grow Light Bulb with 18×2 W 450-460 nm.

Nuclear magnetic resonance (NMR) spectra were acquired on either a Bruker 400 operating at 400 and 100 MHz, a Varian 600 operating at 600 and 150 MHz, or a Bruker 600 operating at 600 and 150 MHz for ¹H and ¹³C, respectively, and are referenced internally according to residual solvent signals. Data for ¹H NMR are recorded as follows: chemical shift (δ, ppm), multiplicity (s, singlet: d, doublet: t, triplet: q, quartet: quint, quintet: m, multiplet), coupling constant (Hz), and integration. Data for ¹³C NMR are reported in terms of chemical shift (δ, ppm). High-resolution mass spectra were obtained using a Thermo Fisher Scientific Q-Exactive HF Orbitrap.

### B. Compound Examples

### Benzyl 5-ethyl-3,6-dihydropyridine-1(2H)-carboxylate (12)

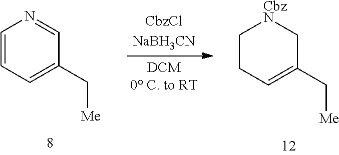 [SMILES: CCc1cccnc1 ; CCC1=CCCN(C(=O)OCc2ccccc2)C1]

A stirred solution of 3-ethylpyridine (10.48 mL, 93.40 mmol, 1.00 equiv.) in DCM/AcOH (374 mL/187 mL, 0.166 M) was cooled to 0° C., and solid sodium cyanoborohydride (14.67 g, 233.00 mmol, 2.50 equiv.) was added in one portion. Benzyl chloroformate (17.26 mL, 121.00 mmol, 1.30 equiv.) was added dropwise over 15 min and the reaction mixture was slowly warmed to ambient temperature over the course of 16 h. The reaction mixture was quenched by the addition of aqueous saturated NaHCO₃ (150 mL) and the organic layers were separated. The aqueous layer was extracted further with DCM (2×200 mL) and the combined organic fractions were dried over sodium sulfate, filtered, and concentrated under reduced pressure. Purification via chromatography on silica gel (20:1 hexanes/ethyl acetate) afforded compound 12 (14.89 g, 65%) as a colorless oil. R_{f}=0.65 (7:3 hexanes/EtOAc). ¹H NMR (400 MHz, CDCl₃) δ (ppm) (rotamers observed): 7.40-7.28 (m, 5H), 5.53 (s, 1H), 5.16 (s, 2H), 3.89-3.81 (m, 2H), 3.52 (t, J=5.74 Hz, 2H), 2.12 (s, 2H), 1.98 (s, 2H), 1.03 (t, J=7.4 Hz, 3H). ¹³C NMR (101 MHz, CDCl₃) δ (ppm) (rotamers observed): 155.27, 137.04, 136.89, 128.41, 127.87, 127.83, 118.03, 117.60, 66.90, 66.80, 49.68, 45.92, 44.68, 40.66, 40.39, 30.51, 27.30, 24.91, 24.57, 12.11, 11.21. HRMS (ESI)=m/z [M+H]⁺ calcd. for C₁₅H₂₀NO₂⁺, 246.1498; found, 246.1490.

### Benzyl 3,4-dibromo-3-ethylpiperidine-1-carboxylate (S20)

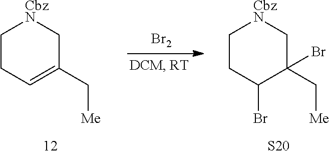 [SMILES: CCC1=CCCN(C(=O)OCc2ccccc2)C1 ; "Br2" ; CCC1(Br)CN(C(=O)OCc2ccccc2)CCC1Br ; "S20"]

Compound S20 was synthesized using a known procedure (Org. Lett. 1 973-976 (1999)). Compound S20 (9.85 g, 96%) was isolated as a clear oil that crystallized into a white solid upon standing. Spectral data matched that reported in the literature. R_{f}=0.45 (7:3 hexanes/EtOAc). ¹H NMR (400 MHz, CDCl₃) δ (ppm) (rotamers observed): 7.41-7.28 (m, 5H), 5.20-5.08 (m, 2H), 4.63-4.59 (s, 1H), 4.31-3.97 (m, 2H) 3.52-3.25 (m, 2H), 2.85-2.70 (m, 1H), 2.08-1.80 (m, 3H), 1.19-1.07 (m, 3H). ¹³C NMR (101 MHz, CDCl₃) δ (ppm) (rotamers observed): 155.42, 155.28, 137.19, 136.67, 128.58, 128.54, 128.15, 128.05, 127.95, 127.85, 71.54, 67.53, 66.96, 55.99, 50.96, 50.59, 49.83, 44.84, 39.52, 39.37, 33.83, 31.52, 31.19, 30.67, 11.38, 8.67.

### Benzyl 5-ethylpyridine-1(2H)-carboxylate (10)

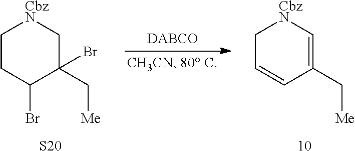 [SMILES: CCC1(Br)CN(C(=O)OCc2ccccc2)CCC1Br ; CCC1=CN(C(=O)OCc2ccccc2)CC=C1 ; "S20" ; "10"]

A 2-neck flask was charged with compound S20 (8.50 g, 20.98 mmol, 1.00 equiv.) and anhydrous DABCO (8.14 g, 72.59 mmol, 3.46 equiv.). The flask was evacuated and refilled with nitrogen 3 times after which anhydrous acetonitrile (159 mL, 0.13 M) was added. The resulting solution was stirred and heated at reflux for 3 h after which the reaction was allowed to cool to ambient temperature and filtered. The reaction vessel and filter cake were washed with DCM (2×50 mL) and the filtrate was concentrated under reduced pressure to remove excess acetonitrile. The resulting residue was diluted in DCM (75 mL) and washed with brine (3×50 mL). The organic fractions were dried over sodium sulfate, filtered, and concentrated under reduced pressure. The residue (3.77 g, 74%) was used immediately in the Diels-Alder reaction without further purification.

### 1-cyclopropylprop-2-en-1-one (9)

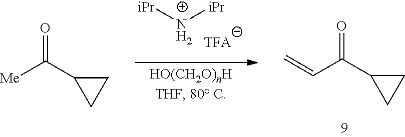

A Schlenk tube was sequentially charged with THF (100 mL, 0.70 M), cyclopropyl methyl ketone (7.00 mL, 70.65 mmol, 1 equiv.), diisopropylammonium trifluoroacetate (17.00 g, 78.99, 1.12 equiv.) and paraformaldehyde (5.00 g, 166.50 mmol, 2.35 equiv.) under a stream of nitrogen. The mixture was stirred and heated at 80° C. for 48 h, after which it was cooled to ambient temperature, diluted with DCM (200 mL) and filtered. The reaction vessel and filter cake were washed with additional DCM. The filtrate was poured into water (200 mL) and the layers were separated. The aqueous layer was further extracted with DCM (2×100 mL) and the combined organic fractions were dried over sodium sulfate, filtered, and concentrated under reduced pressure. Purification via chromatography on silica gel (3:2 hexanes/DCM) afforded compound 9 (5.50 g, 81%) as a light-yellow oil. R_{f}=0.35 (2:1 hexanes/DCM). ¹H NMR (400 MHz, CDCl₃) δ (ppm)=6.43 (dd, J=17.6, 10.5 Hz, 1H), 6.24 (dd, J=17.6, 1.2 Hz, 1H), 5.77 (dd, J=10.5, 1.2 Hz, 1H), 2.20-2.11 (m, 1H), 1.05 (td, J=3.7, 1.0 Hz, 2H), 0.94-0.86 (m, 2H). ¹³C NMR (101 MHz, CDCl₃) δ (ppm)=200.39, 136.57, 127.44, 18.14, 11.12. HRMS (ESI)=m/z [M+H]⁺ calcd. For C₆H₉₀⁺, 97.0658; found, 97.0655.

### Benzyl (1R,4S)-7-(cyclopropanecarbonyl)-6-ethyl-2-azabicyclo[2.2.2]oct-5-ene-2-carboxylate (13)

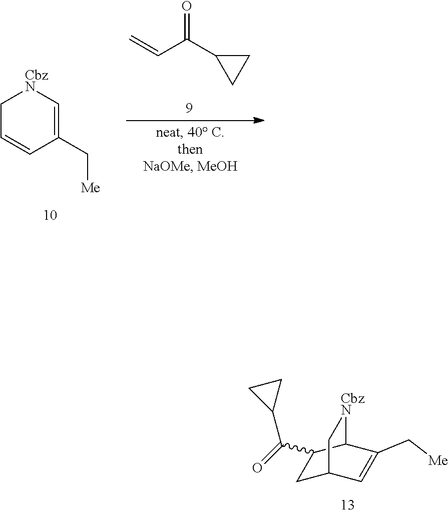

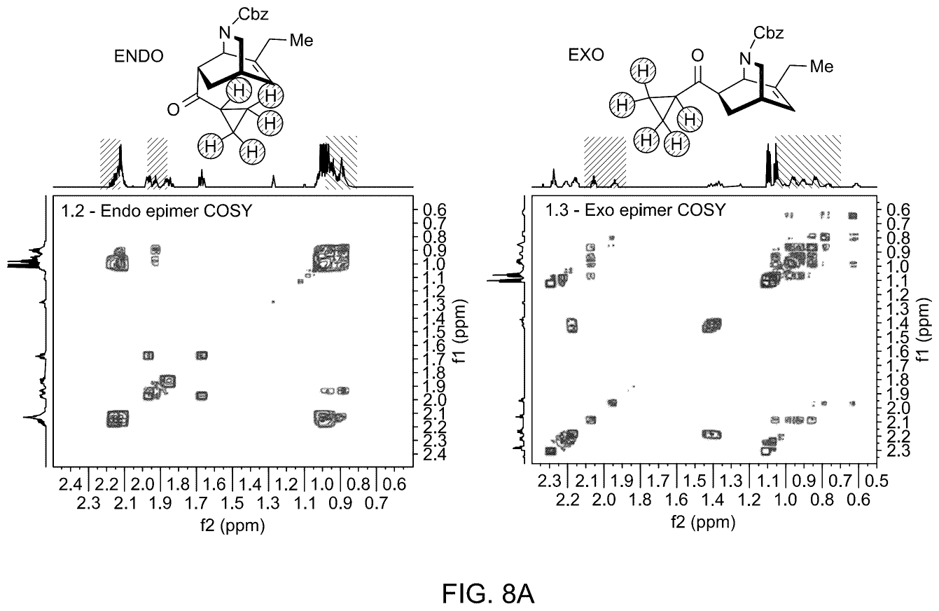

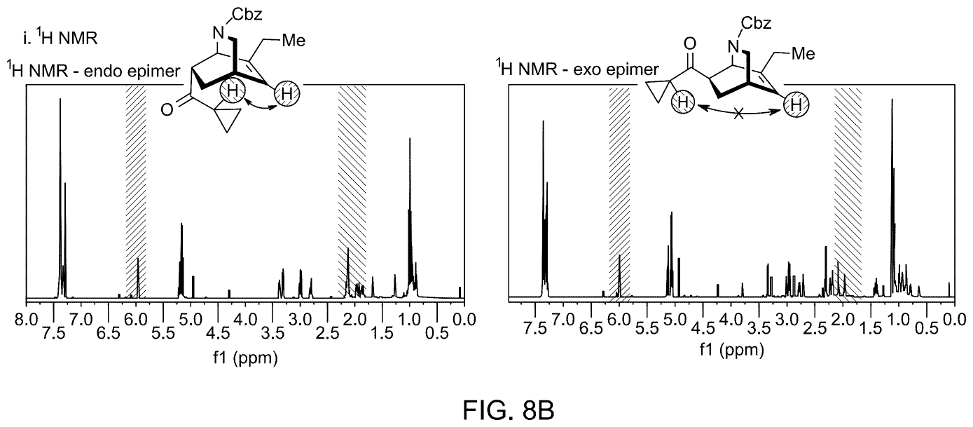

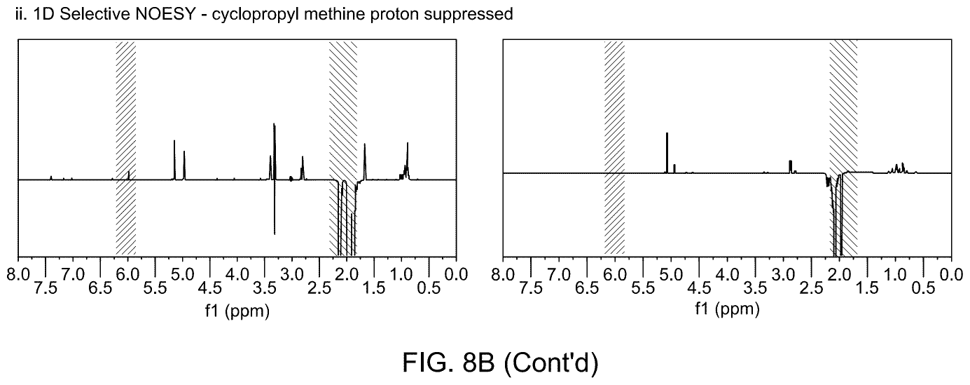

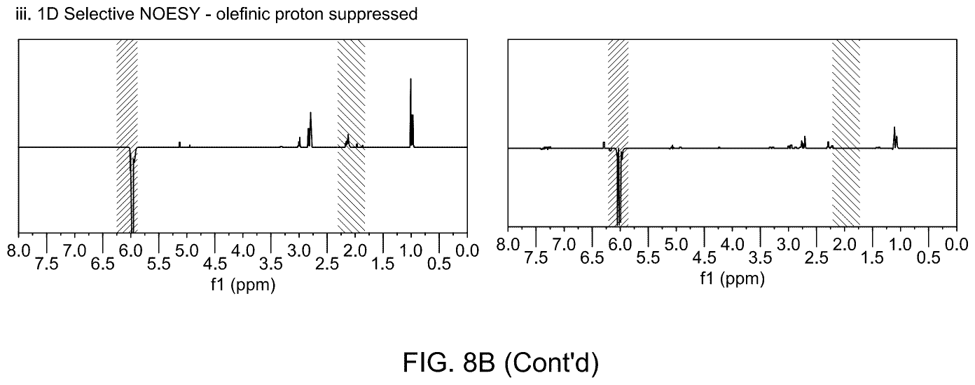

A Schlenk flask was sequentially charged with compound 10 (3.77 g. 15.51 mmol, 1.00 equiv.) and compound 9 (2.98 g, 31.02 mmol, 2.00 equiv.). The mixture was cooled to −78° C. after which it was evacuated and refilled with nitrogen. The mixture was warmed to ambient temperature and this process was repeated 2 times. The degassed mixture was stirred and heated at 40° C. for 72 h. The reaction vessel was cooled to ambient temperature, diluted with methanol (50 mL, 0.30 M) and solid sodium methoxide (0.25 g, 4.65 mmol, 0.30 equiv.) was added in small portions over 5 min. The resulting solution was stirred at ambient temperature for an additional 12 h after which it was concentrated under reduced pressure to remove methanol and any volatiles. The unpurified residue was diluted in DCM (100 mL), poured into water (50 mL) and the layers were separated. The aqueous layer was further extracted with DCM (2×50 mL) and the combined organic extracts were dried over sodium sulfate, filtered, and concentrated under reduced pressure. The residue was purified via chromatography on silica gel (gradient elution 10:1→7:3 hexanes/EtOAc) to afford compound 13 (3.03 g, 64%) as a clear yellow oil and a 50:50 ratio of epimers. Endo and exo epimers of 13 were separated by column chromatography on silica gel using 10:1 DCM/EtOAc and were assigned using a combination of ¹H NMR, COSY, HSQC, and 1D selective gradient NOESY (see FIG. 8A and FIG. 8B). R_{f}=0.40 (7:3 hexanes/ethyl acetate). ¹H NMR (400 MHz, CDCl₃) δ (ppm) (rotamers and epimers observed): 7.38-7.27 (m, 10H), 5.98 (dp, J=7.0, 1.8 Hz, 1H), 5.94 (dt, J=6.4, 1.9 Hz, 1H), 5.19-5.02 (m, 6H), 4.92 (dt, J=8.4, 2.1 Hz, 1H), 3.38-3.24 (m, 3H), 3.02-2.91 (m, 2H), 2.89-2.66 (m, 3H), 2.33-2.19 (m, 2H), 2.19-2.02 (m, 5H), 1.99-1.80 (m, 2H), 1.70-1.61 (m, 2H), 1.39 (dddt, J=16.5, 13.5, 10.7, 3.1 Hz, 1H), 1.14-1.02 (m, 3H), 1.02-0.80 (m, 8H). ¹³C NMR (101 MHz, CDCl₃) δ (ppm) (rotamers and epimers observed): 209.47, 208.93, 208.79, 208.42, 155.14, 154.74, 154.61, 147.27, 146.76, 145.03, 144.77, 137.08, 136.86, 128.52, 128.46, 128.43, 128.38, 128.29, 127.95, 127.89, 127.81, 127.78, 127.64, 127.60, 127.48, 126.94, 125.59, 125.39, 125.04, 124.98, 66.89, 66.84, 66.81, 66.50, 65.34, 52.44, 52.35, 52.24, 51.71, 51.29, 51.04, 51.01, 48.20, 47.85, 47.75, 47.61, 30.61, 30.35, 30.17, 26.58, 26.32, 26.22, 25.90, 24.89, 24.80, 24.69, 19.99, 19.60, 19.44, 19.16, 12.40, 11.73, 11.72, 11.56, 11.45, 11.34, 11.25, 11.17, 11.06. HRMS (ESI)=m/z [M+Na]⁺ calcd. for C₂₁H₂₅NO₃Na⁺, 362.1728; found, 362.1722.

### Benzyl (1R,4S,7S)-7-(cyclopropanecarbonyl)-6-ethyl-2-azabicyclo[2.2.2]oct-5-ene-2-carboxylate (13a)

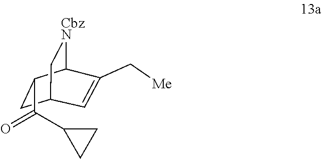 [SMILES: CCC1=CC2CC(C(=O)C3CC3)C1N(C(=O)OCc1ccccc1)C2]

R_{f}=0.40 (7:3 hexanes/ethyl acetate). ¹H NMR (400 MHz, CDCl₃) δ (ppm) (rotamers observed): 7.38-7.27 (m, 5H), 5.93 (dq, J=6.8, 1.9 Hz, 1H), 5.19-5.07 (m, 3H), 3.39-3.21 (m, 2H), 2.97 (tt, J=10.0, 2.6 Hz, 1H), 2.78 (ddt, J=14.6, 6.4, 2.7 Hz, 1H), 2.19-2.02 (m, 3H), 1.98-1.79 (m, 2H), 1.64 (ddd, J=12.3, 9.3, 2.5 Hz, 1H), 1.03-0.80 (m, 7H). ¹³C NMR (101 MHz, CDCl₃) δ (ppm) (rotamers observed)=208.65, 208.28, 155.02, 154.49, 144.93, 144.69, 136.82, 136.76, 128.36, 128.33, 127.84, 127.79, 127.71, 127.67, 124.94, 124.88, 66.78, 66.72, 52.34, 52.14, 50.93, 50.88, 47.75, 47.50, 30.52, 30.27, 26.48, 25.80, 24.82, 19.33, 19.05, 11.44, 11.34, 11.23, 11.15, 11.09. HRMS (ESI)=m/z [M+Na]⁺ calcd. for C₂₁H₂₅NO₃Na⁺, 362.1728; found, 362.1722.

### Benzyl (1R,4S,7R)-7-(cyclopropanecarbonyl)-6-ethyl-2-azabicyclo[2.2.2]oct-5-ene-2-carboxylate (13b)

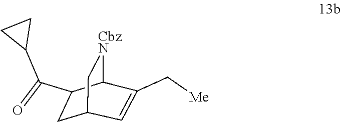 [SMILES: CCC1=CC2CC(C(=O)C3CC3)C1N(C(=O)OCc1ccccc1)C2]

R_{f}=0.40 (7:3 hexanes/ethyl acetate). ¹H NMR (400 MHz, CDCl₃) δ (ppm) (rotamers observed): 7.39-7.28 (m, 5H), 6.00 (m, 1H), 5.10-5.02 (m, 3H), 3.31 (m, 1H), 3.03-2.93 (m, 1H), 2.90-2.71 (m, 2H), 2.35-2.16 (m, 3H), 2.12-1.94 (m, 1H), 1.48-1.35 (m, 1H), 1.14-1.03 (m, 4H), 0.99-0.84 (m, 3H). ¹³C NMR (101 MHz, CDCl₃) δ (ppm) (rotamers observed)=209.49, 208.95, 155.18, 154.77, 147.31, 146.80, 137.13, 136.90, 128.42, 128.33, 127.98, 127.93, 127.68, 127.52, 125.63, 125.43, 66.87, 66.53, 52.47, 52.39, 51.75, 51.32, 48.24, 47.79, 30.39, 30.21, 26.36, 26.27, 24.84, 24.73, 20.03, 19.64, 12.44, 11.77, 11.76, 11.37, 11.23, 11.20, 11.10, 10.83. HRMS (ESI)=m/z [M+Na]⁺ calcd. for C₂₁H₂₅NO₃Na⁺, 362.1728; found, 362.1722.

### Cyclopropyl ((1S,4R,7R)-7-ethyl-2-azabicyclo[2.2.2]octan-6-yl) methanone (14)

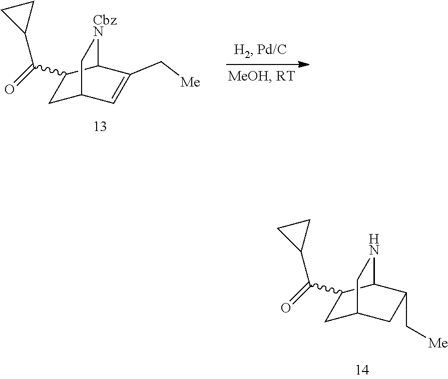 [SMILES: CCC1=CC2CC(C(=O)C3CC3)C1N(C(=O)OCc1ccccc1)C2 ; CCC1CC2CNC1C(C(=O)C1CC1)C2]

A flask was charged with compound 13 (2.95 g, 8.69 mmol, 1.00 equiv.) and unreduced 10 wt % Pd/C (295 mg, 0.27 mmol, 0.03 equiv.). The flask was evacuated and refilled with nitrogen 3 times. MeOH (87 mL, 0.1 M) was added in one portion and hydrogen gas was bubbled through the resulting solution for 1 min. The reaction mixture was stirred under hydrogen atmosphere at ambient temperature for 1 h after which it was filtered through a pad of celite. The reaction vessel and filter cake were washed with DCM (3×50 mL). The filtrate was concentrated under reduced pressure and purified via chromatography on silica gel (10:1 DCM:MeOH, 1% NH₄OH) to afford compound 14 (1.64 g, 91%) as a yellow oil. Based on TLC and LCMS analysis, a 50:50 ratio of C16 epimers is obtained. The epimers were not separated nor characterized individually. R_{f}=0.1 (10:1 DCM/MeOH). ¹H NMR (400 MHz, CDCl₃) δ (ppm) (epimers observed): 3.07-2.88 (m, 4H), 2.88-2.78 (m, 4H), 2.07-1.80 (m, 8H), 1.73-1.65 (m, 2H), 1.64-1.55 (m, 2H), 1.44-1.31 (m, 4H), 1.04-0.98 (m, 4H), 0.98-0.91 (m, 6H), 0.90-0.81 (m, 6H). ¹³C NMR (101 MHz, CDCl₃) δ (ppm) (epimers observed)=213.84, 212.84, 49.36, 49.03 47.43, 47.16, 46.34, 46.24, 39.07, 36.82, 35.08, 34.57, 34.05, 32.34, 27.50, 26.72, 25.06, 19.81, 18.97, 18.48, 11.90, 11.32, 11.25, 10.86, 10.79, 10.76. HRMS (ESI)=m/z [M+H]⁺ calcd. for C₁₃H₂₂NO⁺, 208.1698; found, 208.1699.

### Benzyl (1S,4R,6S,7R)-6-(cyclopropanecarbonyl)-7-ethyl-2-azabicyclo[2.2.2]octane-2-carboxylate (17a)

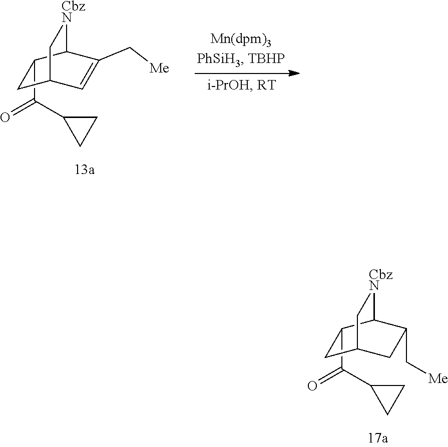 [SMILES: CCC1=CC2CC(C(=O)C3CC3)C1N(C(=O)OCc1ccccc1)C2 ; CCC1CC2CC(C(=O)C3CC3)C1N(C(=O)OCc1ccccc1)C2]

To a stirring solution of 13a (1.50 g, 4.42 mmol, 1.00 equiv.) in anhydrous iPrOH (8.84 mL, 0.50 M) was added phenylsilane (0.82 mL, 6.63 mmol, 1.50 equiv.) and a 5.5 M solution of tert-butyl hydroperoxide in decane (1.20 mL, 6.63 mmol, 1.50 equiv.). The resulting mixture was degassed by bubbling nitrogen through the solution for 10 min. Next, Mn(dpm)₃ (0.27 g, 0.44 mmol, 0.10 equiv.) was added in one portion and the reaction was further degassed for an additional 30 s. The mixture was stirred at ambient temperature for 2 h after which it was concentrated under reduced pressure. The residue was purified via chromatography on silica gel (gradient elution 10:1→7:3 hexanes/EtOAc) to afford compound 17a (1.26 g, 84%) as a clear oil. R_{f}=0.37 (7:3 hexanes/ethyl acetate). ¹H NMR (400 MHz, CDCl₃) δ (ppm) (rotamers observed): 7.40-7.27 (m, 5H), 5.25-4.98 (m, 2H), 4.72-4.35 (m, 1H), 3.46-3.18 (m, 2H), 3.15-2.86 (m, 1H), 2.35 (d, 1H), 2.40-2.16 (m, 1H), 2.03-1.80 (m, 3H), 1.57-1.35 (m, 2H), 1.31-1.21 (m, 1H), 1.05-0.76 (m, 7H). ¹³C NMR (101 MHz, CDCl₃) δ (ppm) (rotamers observed)=210.06, 209.68, 155.15, 154.71, 137.27, 137.05, 128.64, 128.51, 128.09, 127.95, 127.84, 127.77, 127.56, 66.97, 51.62, 51.45, 50.11, 49.76, 48.06, 47.96, 46.38, 40.67, 40.54, 31.87, 31.85, 27.51, 27.48, 26.69, 26.55, 24.92, 24.77, 19.78, 19.57, 12.73, 12.63, 12.46, 12.26, 10.92. HRMS (ESI)=m/z [M+Na]⁺ calcd. For C₂₁H₂₇NO₃Na⁺, 364.1888; found, 364.1889.

### (1S,4R,7R)-6-(4-bromobutanoyl)-7-ethyl-2-azabicyclo[2.2.2]octan-2-ium Bromide (S21)

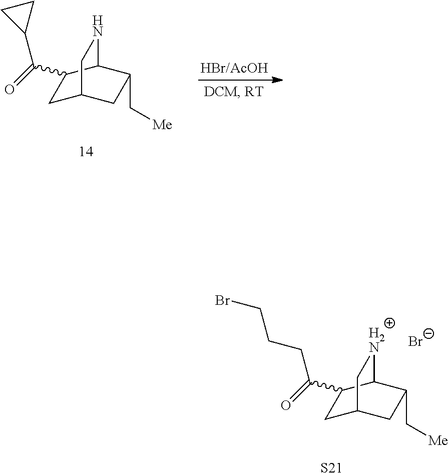

To a stirring solution of compound 14 (1.25 g, 6.03 mmol, 1.00 equiv.) in DCM (6.03 mL, 1.0 M) was added 33 wt % HBr/AcOH (3.12 mL, 18.08 mmol, 3.00 equiv.) in one portion. The solution was stirred at ambient temperature for 2 h after which it was concentrated under reduced pressure. The residue was dried under high vacuum for 1 h and then stirred vigorously in diethyl ether (30 mL). The diethyl ether was decanted and the residue was dried under high vacuum. Product S21 (2.18 g, 98%) was isolated as an orange foam and was used in the next reaction without any further purification or characterization.

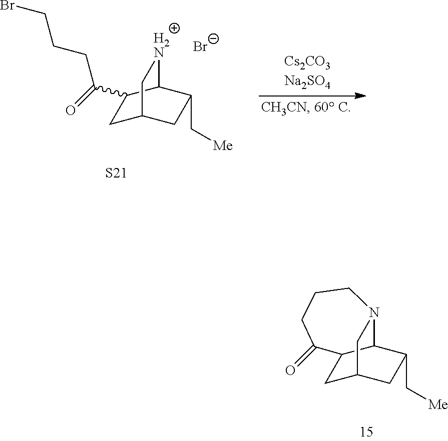

A flask was charged with compound S21 (2.18 g, 5.90 mmol, 1.00 equiv.), anhydrous Cs₂CO₃ (2.88 g, 8.85 mmol, 1.5 equiv.), and anhydrous Na₂SO₄ (2.52 g, 17.71 mmol, 3.00 equiv.). The flask was evacuated and refilled with nitrogen 3 times after which anhydrous CH₃CN (59 mL, 0.10 M) was added in one portion. The reaction mixture was heated to 60° C., and stirred for 6 h. The heterogeneous mixture was cooled to ambient temperature and filtered. The reaction vessel and filter cake were washed with DCM (2×30 mL). The filtrate was concentrated under reduced pressure and the residue was purified via chromatography on silica gel (20:1 DCM/MeOH) to afford compound 15 (0.50 g, 41%) as a light brown oil. R_{f}=0.43 (20:1 DCM/MeOH). ¹H NMR (400 MHz, CDCl₃) δ (ppm)=3.18-2.92 (m, 3H), 2.80 (dd, J=13.2, 2.7 Hz, 1H), 2.66-2.51 (m, 3H), 2.06-1.75 (m, 7H), 1.61-1.49 (m, 1H), 1.39-1.19 (m, 2H), 1.08 (dd, J=12.7, 2.6 Hz, 1H), 0.90 (t, J=7.4 Hz, 3H). ¹³C NMR (101 MHz, CDCl₃) δ (ppm)=216.98, 54.88, 53.83, 50.10, 46.87, 43.79, 41.70, 31.40, 30.54, 27.92, 26.77, 21.24, 12.31. HRMS (ESI)=m/z [M+H]⁺ calcd. For C₁₃H₂₂NO⁺, 208.1698; found, 208.1694.

### Example 1: Epiibogaine (16)

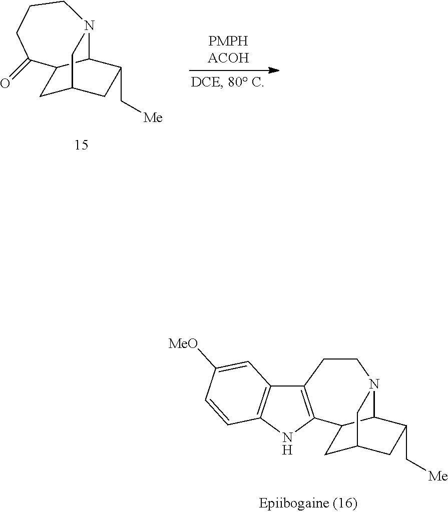 [SMILES: CCC1CC2CC3C(=O)CCCN(C2)C13 ; CCC1CC2CC3c4[nH]c5ccc(OC)cc5c4CCN(C2)C13]

A flask was charged with compound 15 (0.20 g, 0.96 mmol, 1 equiv.) and para-methoxy phenylhydrazine (0.25 g. 1.44 mmol, 1.5 equiv.). The flask was evacuated and refilled with nitrogen 3 times after which anhydrous DCE (9.64 mL, 0.1 M) and AcOH (0.83 mL, 14.47 mmol, 15 equiv.) was added. The resulting mixture was degassed by bubbling nitrogen through the solution for 10 min. The flask was heated to 80° C., and stirred for 12 h after which it was cooled to ambient temperature and diluted with DCM (20 mL). Saturated aqueous NaHCO₃ was added to the reaction mixture until the pH was adjusted to 7-8. The organic layer was separated, and the aqueous layer was further extracted with DCM (2×50 mL). The combined organic extracts were dried over sodium sulfate, filtered and concentrated under reduced pressure. Purification via chromatography on silica gel (gradient elution 20:1→10:1 DCM/MeOH, 0.25% NH₄OH) afforded epiibogaine (0.24 g, 81%) as a colorless oil. R_{f}=0.33 (10:1 DCM/MeOH). ¹H NMR (400 MHz, MeOD) δ (ppm)=7.16 (d, J=8.7 Hz, 1H), 6.95 (d, J=2.4 Hz, 1H), 6.73 (dd, J=8.8, 2.4 Hz, 1H), 3.81 (s, 3H), 3.60-3.53 (m, 2H), 3.55-3.41 (m, 3H), 3.41-3.37 (m, 1H), 3.35-3.23 (m, 1H), 3.16-3.06 (m, 1H), 2.28 (d, J=2.6 Hz, 1H), 2.22-2.01 (m, 3H), 1.75-1.63 (m, 1H), 1.56 (td, J=7.3, 3.1 Hz, 2H), 1.38-1.19 (m, 1H), 1.01 (t, J=7.4 Hz, 3H). ¹³C NMR (101 MHz, MeOD) δ (ppm)=154.01, 139.67, 130.07, 128.55, 111.18, 111.01, 107.34, 99.39, 59.08, 56.25, 54.92, 50.37, 37.60, 32.16, 29.14, 28.18, 26.49, 24.11, 17.90, 10.58. HRMS (ESI)=m/z [M+H]⁺ calcd. For C₂₀H₂₇N₂O⁺, 311.2128; found, 311.2126.

### Benzyl pyridine-1(2H)-carboxylate (24)

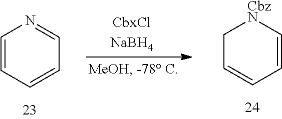

A flask was charged with methanol (500 mL, 0.50 M) and pyridine (20.36 mL, 252.84 mmol, 1.00 equiv.) and cooled to −78° C. after which sodium borohydride (11.47 g, 303.41 mmol, 1.20 equiv.) was added in one portion. Benzyl chloroformate (43.13 mL, 303.41 mmol, 1.20 equiv.) was added dropwise over 1 h to the reaction mixture. The reaction mixture was stirred at −78° C. for an additional 3 h after which it was diluted in Et₂O (200 mL), poured into 1 M HCl (400 mL) and the layers were separated. The aqueous layer was extracted with Et₂O (2×200 mL) and the combined organic extracts were washed with 1 M NaOH (100 mL) followed by brine (100 mL). The organic extracts were dried over sodium sulfate, filtered, and concentrated under reduced pressure. The residue (54.31 g, 98%) was immediately used without further purification or characterization.

### Benzyl (1S,4S)-7-(cyclopropanecarbonyl)-2-azabicyclo[2.2.2]oct-5-ene-2-carboxylate (20)

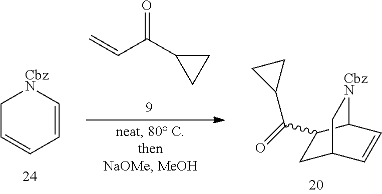

A Schlenk flask was sequentially charged with compound 24 (15.50 g, 72.00 mmol, 1.00 equiv.) and compound 9 (13.84 g, 144.01 mmol, 2.00 equiv.). The mixture was cooled to −78° C. after which the flask was evacuated and refilled with nitrogen 3 times. The mixture was stirred and heated at 80° C. for 72 h. The reaction vessel was cooled to ambient temperature, diluted with methanol (240 mL, 0.30 M) and solid sodium methoxide (1.16 g, 21.60 mmol, 0.30 equiv.) was added in small portions over 5 min. The resulting solution was stirred at ambient temperature for an additional 12 h after which it was concentrated under reduced pressure to remove methanol and any volatiles. The unpurified residue was diluted in DCM (200 mL), poured into water (100 mL) and the layers were separated. The aqueous layer was further extracted with DCM (2×100 mL) and the combined organic fractions were dried over sodium sulfate, filtered, and concentrated under reduced pressure. The residue was purified via chromatography on silica gel (7:3 hexanes/EtOAc) to afford compound 17 (20.17 g. 90%) as a clear yellow oil. Compound 20 was isolated as a 50:50 mixture of exo:endo epimers.

Separation of endo and exo epimers can be achieved by chromatography on silica gel using 10:1 DCM/EtOAc.

### Benzyl (1S,4S,7R)-7-(cyclopropanecarbonyl)-2-azabicyclo[2.2.2]oct-5-ene-2-carboxylate

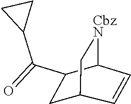 [SMILES: O=C(C1CC1)C1CC2C=CC1N(C(=O)OCc1ccccc1)C2]

R_{f}=0.42 (7:3 hexanes:EtOAc). ¹H NMR (400 MHz, CDCl₃) δ (ppm) (rotamers and epimer observed): 7.38-7.30 (m, 10H), 6.58-6.44 (m, 2H), 6.43-6.34 (m, 1H), 6.30-6.24 (m, 1H), 5.23 (d, J=6.1 Hz, 1H), 5.18-5.02 (m, 4H), 4.70 (d, J=5.7 Hz, 1H), 3.38-3.25 (m, 3H), 3.05-2.71 (m, 5H), 2.27-2.16 (m, 1H), 2.14-2.02 (m, 1H), 2.03-1.81 (m, 2H), 1.77-1.67 (1H), 1.48-1.41 (m, 1H), 1.06-0.81 (m, 8H). ¹³C NMR (101 MHz, CDCl₃) δ (ppm) (rotamers and epimer observed): 209.36, 208.75, 208.49, 155.26, 154.76, 137.04, 136.85, 136.78, 135.52, 135.35, 134.90, 134.72, 132.28, 131.88, 130.42, 130.15, 128.53, 128.50, 128.43, 128.36, 128.18, 128.09, 127.99, 127.84, 127.72, 127.57, 127.49, 66.93, 66.59, 52.49, 52.32, 52.29, 47.91, 47.62, 47.50, 47.19, 47.00, 30.78, 30.55, 30.31, 30.13, 24.28, 23.62, 23.50, 20.05, 19.73, 19.43, 19.27, 12.50, 11.82, 11.50, 11.36, 11.21, 10.86, 10.64. HRMS (ESI)=m/z [M+H]⁺ calcd. For C₁₉H₂₂NO₃⁺, 312.1598; found, 312.1599.

### Benzyl (1S,4S,7S)-7-(cyclopropanecarbonyl)-2-azabicyclo[2.2.2]oct-5-ene-2-carboxylate

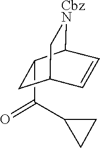 [SMILES: O=C(C1CC1)C1CC2C=CC1N(C(=O)OCc1ccccc1)C2]

R_{f}=0.35 (10:1 DCM:EtOAc). ¹H NMR (400 MHz, CDCl₃) δ (ppm) (rotamers observed): 7.39-7.30 (m, 5H), 6.41-6.37 (m, 1H), 6.30-6.25 (m, 1H), 5.29 (dt, J=6.0, 2.0 Hz, 1H), 5.15 (d, J=12.4 Hz, 2H), 3.41-3.22 (m, 2H), 3.02 (ddd, J=10.2, 6.8, 4.1 Hz, 1H), 2.85 (d, J=15.8 Hz, 1H), 2.05-1.68 (m, 3H), 1.07-0.81 (m, 4H). ¹³C NMR (101 MHz, CDCl₃) δ (ppm) (rotamers observed): 208.48, 155.24, 136.81, 134.89, 134.69, 130.39, 130.11, 128.47, 127.95, 66.90, 52.62, 52.27, 47.18, 46.97, 30.75, 30.51, 25.18, 24.23, 19.41, 19.23, 11.49, 11.19, 10.85, 10.61. HRMS (ESI)=m/z [M+H]⁺ calcd. For C₁₉H₂₂NO₃⁺, 312.1598; found, 312.1599.

The endo epimer was assigned based on the predominant isomer obtained following the Diels-Alder reaction prior to epimerization.

### 3-((1S,4R)-2-((benzyloxy) carbonyl)-7-(cyclopropanecarbonyl)-2-azabicyclo[2.2.2]octan-6-yl) propanoic Acid (25)

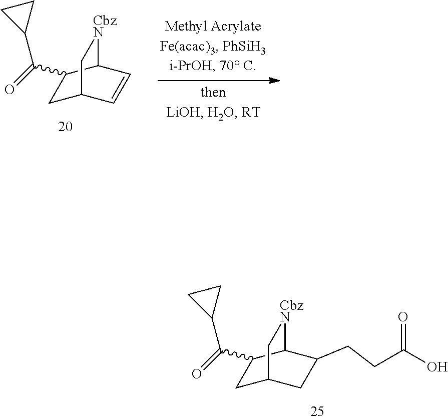 [SMILES: O=C(C1CC1)C1CC2C=CC1N(C(=O)OCc1ccccc1)C2 ; O=C(O)CCC1CC2CC(C(=O)C3CC3)C1N(C(=O)OCc1ccccc1)C2]

A flask was sequentially charged with compound 20 (3.30 g, 10.59 mmol, 1.00 equiv.), isopropanol (53 mL, 0.2 M), methyl acrylate (5.76 mL, 63.58 mmol, 6.00 equiv.), and Fe(acac)₃ (1.12 g, 3.18 mmol, 0.30 equiv.). To this solution was added phenylsilane (3.91 mL, 31.79 mmol, 3.00 equiv.) in one portion. The mixture was stirred and heated at 70° C. under air atmosphere for 24 h, after which it was cooled to ambient temperature. The reaction mixture was diluted in H₂O (100 mL) and LiOH (2.53 g, 105.97 mmol, 10.00 equiv.) was added in one portion. The solution was stirred at ambient temperature for 12 h after which it was acidified with 1 M HCl (100 mL). The mixture was poured into DCM (200 mL) and the layers were separated. The aqueous layer was further extracted with DCM (2×100 mL) and the combined organic fractions were dried over sodium sulfate, filtered, and concentrated under reduced pressure. The residue was purified via chromatography on silica gel (gradient elution 10:1→1:1 hexanes/EtOAc) to afford compound 25 (3.02 g, 74%) as a clear foam.

The configuration at C20 was determined retroactively upon completing the total synthesis of ibogaine. Based on LCMS analysis, we presume that a 50:50 ratio of C16 epimers is obtained. Vigorous bubbling is observed upon 5 min of heating. Purification of 25 can be challenging when using chromatography on silica gel as the compound streaks. Alternatively, the methyl ester can be chromatographed easily (gradient elution 10:1→7:3 hexanes/EtOAc) and then a subsequent hydrolysis can yield compound 25 with no purification needed.

R_{f}=0.15 (7:3 hexanes/EtOAc). ¹H NMR (400 MHz, CDCl₃) δ (ppm) (rotamers and epimers observed): 7.38-7.27 (m, 5H), 5.20-4.96 (m, 2H), 4.70-4.36 (m, 1H), 3.54-3.15 (m, 2H), 3.06-2.85 (m, 1H), 2.59-2.16 (m, 4H), 2.11-1.84 (m, 3H), 1.81-1.41 (m, 4H), 1.17-0.66 (m, 4H). ¹³C NMR (101 MHz, CDCl₃) δ (ppm) (rotamers and epimers observed): 210.17, 209.45, 178.74, 156.21, 137.09, 128.62, 128.56, 128.52, 128.33, 128.20, 127.92, 127.69, 127.66, 127.62, 67.23, 66.86, 52.14, 48.95, 48.74, 37.79, 37.68, 33.64, 32.26, 31.99, 31.53, 31.39, 30.02, 29.88, 29.69, 26.06, 25.96, 24.14, 24.03, 19.99, 19.88, 19.62, 12.55, 11.66, 11.36, 11.20. HRMS (ESI)=m/z [M+H]⁺ calcd. For C₂₂H₂₈NO₅⁺, 387.1968; found, 387.1962.

### Benzyl (1S,4R)-6-(cyclopropanecarbonyl)-7-ethyl-2-azabicyclo[2.2.2]octane-2-carboxylate (26)

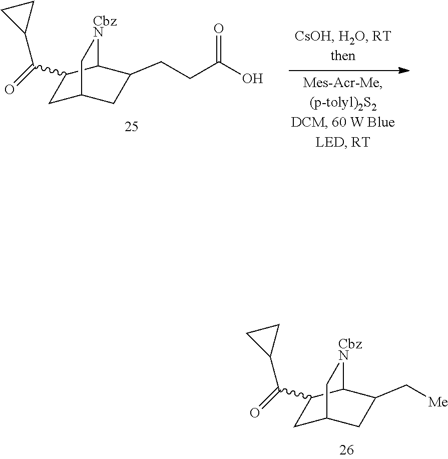 [SMILES: O=C(O)CCC1CC2CC(C(=O)C3CC3)C1N(C(=O)OCc1ccccc1)C2 ; CCC1CC2CC(C(=O)C3CC3)C1N(C(=O)OCc1ccccc1)C2]

A glass reaction tube was charged with compound 25 (1.85 g. 4.79 mmol, 1.00 equiv.). A solution of CsOH·H₂O (806 mg, 4.79 mmol, 1.00 equiv.) in H₂O (9.6 mL) was added and the milky white heterogeneous mixture was stirred vigorously for 1 h. In a separate flask, 9-mesityl-10-methylacridinium tetrafluoroborate (96 mg, 0.23 mmol, 0.05 equiv.) and p-tolyl disulfide (1.77 g. 7.19 mmol, 1.50 equiv.) was dissolved in DCM (38.4 mL). The acridinium/disulfide solution was added to compound 25 in one portion. The biphasic solution was tightly capped under air atmosphere and stirred (>1000 rpm) between two 30 W blue LED lamps (8 cm distance between reaction vessel and lamp) for 24 h at ambient temperature. After 24 h, another portion of 9-mesityl-10-methylacridinium tetrafluoroborate (96 mg, 0.23 mmol, 0.05 equiv.) was added to the reaction mixture and the resulting solution was placed in the photoreactor for an additional 24 h. Upon reaction completion, the reaction mixture was poured into water (50 mL) and the layers were separated. The aqueous layer was further extracted with DCM (2×50 mL) and the combined organic extracts were dried over sodium sulfate, filtered, and concentrated under reduced pressure. The residue was purified via chromatography on silica gel (7:3 hexanes/EtOAc) to afford compound 26 as an orange oil. Compound 26 was further purified by dissolution in ethyl acetate and subsequent addition of activated charcoal (50 mg). Heating the stirred solution at 50° C. for 30 min, followed by filtration and concentration under reduced pressure afforded compound 26 (1.16 g, 71%) as a light-yellow oil. R_{f}=0.65 (7:3 hexanes/EtOAc). ¹H NMR (400 MHz, CDCl₃) δ (ppm) (rotamers and epimers observed): 7.40-7.27 (m, 5H), 5.27-4.97 (m, 2H), 4.70-4.37 (m, 1H), 3.46-3.11 (m, 2H), 3.03-2.84 (m, 1H), 2.38-2.17 (m, 1H), 2.13-1.63 (m, 3H), 1.54-1.16 (m, 4H), 1.16-1.06 (m, 1H), 1.05-0.72 (m, 7H). ¹³C NMR (101 MHz, CDCl₃) δ (ppm) (rotamers and epimers observed): 210.37, 209.65, 156.00, 155.72, 137.35, 136.94, 128.48, 128.21, 127.80, 127.73, 66.95, 66.55, 52.44, 51.78, 51.08, 50.30, 49.35, 49.03, 46.88, 46.41, 46.31, 43.78, 40.78, 40.59, 36.50, 36.34, 36.03, 35.96, 35.40, 35.16, 34.87, 34.72, 32.38, 32.23, 29.53, 28.28, 28.14, 27.90, 26.58, 26.43, 26.15, 26.05, 24.26, 24.15, 19.88, 19.57, 12.41, 11.77, 11.75, 11.24, 11.09. HRMS (ESI)=m/z [M+H]⁺ calcd. For C₂₁H₂₈NO₃⁺, 342.2068; found, 342.2065.

### Example 2: General Procedure for Preparation of Iboga Derivatives

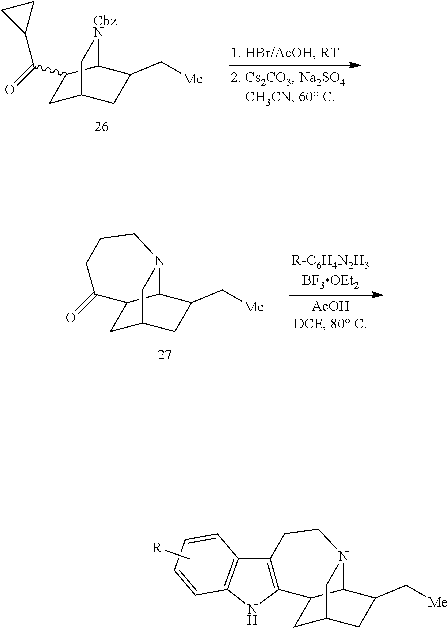

A solution of 33 wt % HBr/AcOH (3.00 equiv.) was added in one portion to compound 26 (1.00 equiv.) while stirring. The dark orange mixture was stirred at ambient temperature for 2 h after which it was concentrated under reduced pressure. The residue was dried under high vacuum for 1 h and then stirred vigorously in diethyl ether (30-50 mL). The diethyl ether was decanted, and the resulting alkyl bromide was dried under high vacuum. This process was repeated 3× to ensure all the benzyl bromide was removed. A flask was charged with alkyl bromide (1.00 equiv.), anhydrous Cs₂CO₃ (1.50 equiv.), and anhydrous Na₂SO₄ (3.00 equiv.). The flask was evacuated and refilled with nitrogen 3 times after which anhydrous CH₃CN (0.1 M) was added in one portion. The reaction mixture was heated to 60° C., and stirred for 6 h. The heterogeneous mixture was cooled to ambient temperature and filtered. The reaction vessel and filter cake were washed with DCM (2×10 mL). The filtrate was concentrated under reduced pressure and the resulting residue was dissolved in DCM (10 mL) and washed with sat. NaHCO₃ (3×5 mL). The organic extract was dried over sodium sulfate, filtered, and concentrated under reduced pressure to yield compound 27 as an unpurified brown oil.

A flask was charged with unpurified compound 27 (1.00 equiv.) and substituted phenylhydrazine (1.50 equiv.). The flask was evacuated and refilled with nitrogen 3 times after which anhydrous DCE (0.1 M) and AcOH (15.00 equiv.) was added. The resulting mixture was degassed by bubbling nitrogen through the solution for 10 min. The flask was heated to 80° C., and stirred for 1 h after which it was cooled to ambient temperature and BF₃·OEt₂ (1.20 equiv.) was added in one portion. The resulting mixture was heated at 80° C. for 12 h after which it was cooled to ambient temperature and diluted with DCM (10 mL). Saturated aqueous NaHCO₃ was added to the reaction mixture until the pH was adjusted to 7-8. The organic layer was separated, and the aqueous layer was further extracted with DCM (2×20 mL). The combined organic extracts were dried over sodium sulfate, filtered and concentrated under reduced pressure. Purification via chromatography on silica gel (gradient elution 20:1→10:1 DCM/MeOH, 0.25% NH₄OH) afforded iboga derivates.

### Example 3: Ibogaine (1)—500 mg Scale

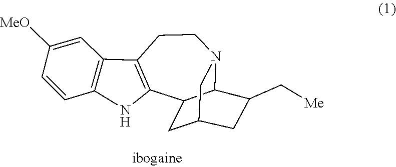 [SMILES: CCC1CC2CC3c4[nH]c5ccc(OC)cc5c4CCN(C2)C13]

Synthesized from compound 26 (500 mg, 1.46 mmol, 1.00 equiv.) following the general procedure for iboga derivative preparation. Para-methoxyphenylhydrazine·HCl (382 mg. 2.19 mmol, 1.50 equiv.) was used and BF₃·OEt₂ was omitted from the Fischer indole reaction. Ibogaine (140 mg, 31% over three steps) was isolated as an orange foam.

### Example 4: Ibogaine (1)—1.33 g Scale

Synthesized from compound 26 (1.33 g, 3.86 mmol, 1 equiv.) following the general procedure for iboga derivative preparation. Para-methoxyphenylhydrazine·HCl (1.02 g, 5.84 mmol, 1.5 equiv.) was used and BF₃·OEt₂ was omitted from the Fischer indole reaction. Ibogaine (263 mg, 22% over three steps) was isolated as an orange foam. R_{f}=0.33 (10:1 DCM/MeOH). ¹H NMR (400 MHz, CDCl₃) δ (ppm)=7.53 (s, 1H), 7.14 (d, J=8.7 Hz, 1H), 6.93 (m, 1H), 6.77 (dd, J=8.6, 2.4 Hz, 1H), 3.86 (s, 3H), 3.41-3.30 (m, 2H), 3.17-3.10 (m, 1H), 3.09-3.05 (m, 1H), 3.00-2.95 (m, 1H), 2.93-2.87 (m, 1H), 2.85 (s, 1H), 2.61 (dd, J=17.3, 5.4 Hz, 1H), 2.09-2.00 (m, 1H), 1.85 (s, 1H), 1.83-1.77 (m, 1H), 1.65 (dq, J=13.3, 3.5 Hz, 1H), 1.55 (q, J=7.2 Hz, 2H), 1.48-1.43 (m, 1H), 1.24-1.18 (m, 1H), 0.90 (t, J=7.1 Hz, 3H). ¹³C NMR (101 MHz, CDCl₃) δ (ppm)=153.98, 142.89, 130.13, 129.71, 110.75, 110.69, 109.17, 100.39, 57.48, 56.02, 54.19, 49.98, 41.97, 41.61, 34.22, 32.11, 27.84, 26.51, 20.71, 11.91. HRMS (ESI)=m/z [M+H]⁺ calcd. For C₂₀H₂₇N₂O⁺, 311.2128; found, 311.2128. IR (Smart iTX Diamond)=v 3397, 3058, 2917, 2854, 1624, 1585, 1452, 1433, 1361, 790 cm⁻¹.

Enantiopure (+)-ibogaine was synthesized from intermediate (−)—S19. Enantiopure (−)-ibogaine was synthesized from intermediate (+)—S19. Enantiopurity determined by HPLC. Specific Rotation for (+)-ibogaine=[α]_{D}²⁰=50.1 (c=0.01 in MeOH). Enantomeric Excess (ee)=>99% (determined by chiral HPLC).

### Example 5: Ibogamine (2)

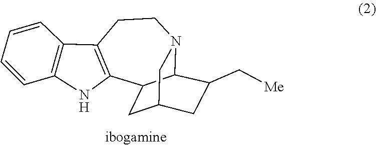 [SMILES: CCC1CC2CC3c4[nH]c5ccccc5c4CCN(C2)C13]

Synthesized from compound 26 (500 mg, 1.46 mmol, 1 equiv.) following the general procedure for iboga derivative preparation. Phenylhydrazine·HCl (317 mg, 2.19 mmol, 1.50 equiv) was used in the Fischer indole reaction. Ibogamine (118 mg, 29% over 3 steps) was isolated as a yellow foam. R_{f}=0.37 (10:1 DCM/MeOH). ¹H NMR (400 MHz, CDCl₃) δ (ppm)=7.66 (br. s., 1H), 7.48-7.44 (dd, J=6.95 Hz, 1.61 Hz, 1H), 7.27-7.24 (m, 1H), 7.14-7.05 (m, 2H), 3.47-3.28 (m, 2H), 3.22-3.12 (dt, J=13.61 Hz, 3.71 Hz, 1H), 3.10-3.02 (m, 2H), 2.99-2.92 (m, 1H), 2.92-2.87 (m, 1H), 2.76-2.68 (m, 1H), 2.11-2.01 (m, 1H), 1.90-1.78 (m, 2H), 1.69-1.62 (m, 1H), 1.61-1.53 (m, 2H), 1.52-1.45 (m, 1H), 1.26-1.20 (m, 1H), 0.90 (t, J=7.10 Hz, 3H). ¹³C NMR (101 MHz, CDCl₃) δ (ppm)=141.37, 134.67, 129.55, 121.10, 119.18, 117.89, 110.12, 109.04, 57.83, 54.35, 49.95, 41.76, 40.95, 33.97, 31.79, 27.59, 26.23, 20.44, 11.90. HRMS (ESI)=m/z [M+H]⁺ calcd. For C₁₉H₂₅N₂⁺, 281.2018; found, 281.2026. IR (Smart iTX Diamond)=v 3399, 3052, 2920, 2850, 1671, 1602, 1494, 1461, 1362, 735 cm⁻¹.

### Example 6:10-fluoroibogamine (29)

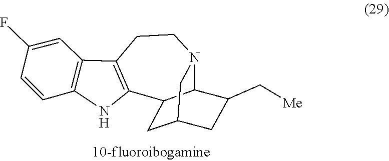 [SMILES: CCC1CC2CC3c4[nH]c5ccc(F)cc5c4CCN(C2)C13]

Synthesized from compound 26 (200 mg, 0.59 mmol, 1.00 equiv.) following the general procedure for iboga derivative preparation, with 4-fluorophenylhydrazine·HCl (143 mg, 0.88 mmol, 1.50 equiv.) being used in the Fischer indole reaction. 10-fluoroibogamine (56 mg, 32% yield over 3 steps) was isolated as a clear orange oil. R_{f}=0.42 (10:1 DCM/MeOH). ¹H NMR (400 MHz, CDCl₃) δ (ppm)=7.64 (s, 1H), 7.15 (dd, J=8.7, 4.4 Hz, 1H), 7.10 (dd, J=9.8, 2.5 Hz, 1H), 6.84 (td, J=9.1, 2.5 Hz, 1H), 3.43-3.27 (m, 2H), 3.20-2.98 (m, 3H), 2.96-2.86 (m, 2H), 2.63-2.54 (m, 1H), 2.10-2.04 (m, 1H), 1.90-1.77 (m, 2H), 1.66 (m, 1H), 1.59-1.46 (m, 3H), 1.22 (m, 1H), 0.90 (t, J=7.1 Hz, 3H). ¹³C NMR (101 MHz, CDCl₃) δ (ppm)=157.89 (¹J_{CF}=234.29 Hz), 143.72, 131.08, 130.11, 110.58 (¹J_{CF}=9.60 Hz), 109.53, 109.01 (¹J_{CF}=26.29 Hz), 103.03 (¹J_{CF}=22.72 Hz), 57.53, 54.19, 50.01, 41.89, 41.38, 34.08, 31.95, 27.77, 26.38, 20.59, 11.92. HRMS (ESI)=m/z [M+H]⁺ calcd. For C₁₉H₂₄N₂F⁺, 299.1928; found, 299.1925. IR (Smart iTX Diamond)=v 3397, 3051, 2923, 2856, 1671, 1580, 1485, 1454, 1362, 734 cm⁻¹.

Enantiopure (+)-fluoroibogamine was synthesized from intermediate (−)—S19. Enantiopure (−)-fluoroibogamine was synthesized from intermediate (+)—S19. Specific Rotation for (−)-fluoroibogamine=[α]_{D}²⁰=−35.3 (c=0.01 in MeOH). Specific Rotation for (+)-fluoroibogamine=[α]_{D}²⁰=34.6 (c=0.01 in MeOH).

### Example 7: Tabernanthine (4)

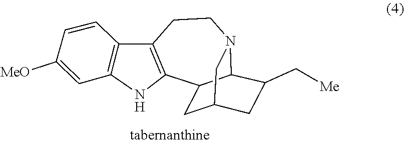 [SMILES: CCC1CC2CC3c4[nH]c5cc(OC)ccc5c4CCN(C2)C13]

Synthesized from compound 26 (200 mg, 0.59 mmol, 1.00 equiv.) following the general procedure for iboga derivative preparation, with 3-methoxyphenylhydrazine HCl (153 mg, 0.88 mmol, 1.5 equiv.) being used in the Fischer indole reaction. Tabernanthine (24 mg, 13.3% yield over 3 steps) was isolated as a clear orange oil. R_{f}=0.35, 1H NMR (400 MHz, CDCl₃) δ (ppm)=7.51 (s, 1H), 7.35 (d, J=8.5 Hz, 1H), 6.77 (s, 1H) 6.76 (d, J=8.5 Hz, 1H), 3.83 (s, 3H), 3.44-3.38 (m, 1H), 3.36-3.26 (m, 1H), 3.16 (td, J=13.1, 3.7 Hz, 1H), 3.05 (d, J=10.4 Hz, 2H), 2.95-2.87 (m, 2H), 2.66 (d, J=16.3 Hz, 1H), 2.07-2.00 (m, 1H), 1.87-1.77 (m, 2H), 1.67-1.51 (m, 4H), 1.24-1.20 (m, 1H), 0.90 (t, J=7.1 Hz, 3H). ¹³C NMR (101 MHz, CDCl₃) δ (ppm)=155.86, 140.47, 135.35, 124.19, 118.46, 108.83, 108.48, 94.37, 57.77, 55.84, 54.11, 49.82, 41.88, 41.28, 34.19, 32.06, 27.67, 26.45, 20.72, 11.89. HRMS (ESI)=m/z [M+H]⁺ calcd. For C₂₀H₂₇N₂O⁺, 311.2128; found, 311.2127. IR (Smart iTX Diamond)=v 3356, 2952, 2918, 2855, 1626, 1564, 1496, 1460, 1380, 735 cm⁻¹.

### Example 8:12-methoxyibogamine (28)

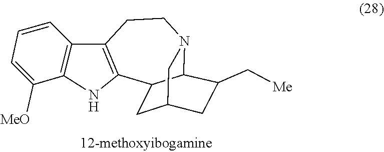 [SMILES: CCC1CC2CC3c4[nH]c5c(OC)cccc5c4CCN(C2)C13]

Synthesized from compound 26 (200 mg, 0.59 mmol, 1.00 equiv.) following the general procedure for iboga derivative preparation, with 2-methoxyphenylhydrazine·HCl (153 mg, 0.88 mmol, 1.50 equiv.) was used in the Fischer indole reaction. 12-methoxyibogamine (32 mg, 17.4% yield over 3 steps) was isolated as a clear yellow oil. R_{f}=0.31 (10:1 DCM/MeOH). ¹H NMR (400 MHz, CDCl₃) δ (ppm)=7.85 (s, 1H), 7.09 (d, J=7.9 Hz, 1H), 7.00 (t, J=7.8 Hz, 1H), 6.59 (dd, J=7.7, 0.9 Hz, 1H), 3.94 (s, 3H), 3.41-3.31 (m, 2H), 3.19-3.05 (m, 2H), 3.02-2.92 (m, 2H), 2.86 (d, J=2.0 Hz, 1H), 2.69-2.62 (m, 1H), 2.08-2.00 (m, 1H), 1.86-1.78 (m, 2H), 1.67-1.47 (m, 4H), 1.21-1.17 (m, 1H), 0.90 (t, J=7.1 Hz, 3H). ¹³C NMR (101 MHz, CDCl₃) δ (ppm)=145.49, 141.51, 131.02, 124.72, 119.47, 110.89, 109.74, 101.25, 57.55, 55.33, 54.21, 49.97, 41.94, 41.47, 34.23, 32.10, 27.81, 26.51, 20.84, 11.89. HRMS (ESI)=m/z [M+H]⁺ calcd. For C₂₀H₂₇N₂O⁺, 311.2128; found, 311.2127. IR (Smart iTX Diamond)=v 3398, 3049, 2919, 2854, 1684, 1577, 1499, 1456, 1386, 729 cm⁻¹.

### Example 9: Ibogaline (3)

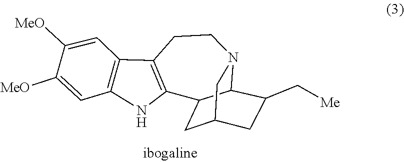 [SMILES: CCC1CC2CC3c4[nH]c5cc(OC)c(OC)cc5c4CCN(C2)C13]

Synthesized from compound 26 (200 mg, 0.59 mmol, 1.00 equiv.) following the general procedure for iboga derivative preparation, with 3,4-dimethoxyphenylhydrazine·HCl (179 mg, 0.88 mmol, 1.50 equiv.) being used in the Fischer indole reaction. Ibogaline (32 mg, 16.4% yield over 3 steps) was isolated as a brown oil. R_{f}=0.22 (10:1 DCM/MeOH). ¹H NMR (400 MHz, CDCl₃) δ (ppm)=8.50 (s, 1H), 6.86 (s, 1H), 6.84 (s, 1H), 3.91 (s, 3H), 3.86 (s, 3H), 3.47-3.35 (m, 3H), 3.22-2.93 (m, 5H), 2.38-2.26 (m, 1H), 2.00-1.83 (m, 3H), 1.77-1.68 (m, 1H), 1.57-1.37 (m, 3H), 0.89 (t, J=7.24, 3H). ¹³C NMR (101 MHz, CDCl₃) δ (ppm)=147.19, 145.45, 137.15, 128.90, 120.90, 108.05, 99.82, 94.93, 60.54, 56.62, 56.45, 56.38, 47.28, 34.20, 33.36, 27.91, 26.93, 21.19, 18.64, 14.33, 11.93. HRMS (ESI)=m/z [M+H]⁺ calcd. For C₂₁H₂₉N₂O₂⁺, 341.2228; found, 341.2231. IR (Smart iTX Diamond)=v 3339, 3053, 2931, 2874, 1631, 1566, 1484, 1463, 730 cm⁻¹.

### ((1S,4R)-2-azabicyclo[2.2.2]octan-6-yl) (cyclopropyl) methanone (S4)

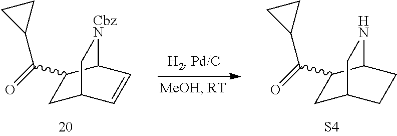

A flask was charged with compound 20 (10.30 g, 33.07 mmol, 1.00 equiv.) and unreduced 10 wt % Pd/C (1.03 g, 0.96 mmol, 0.03 equiv.). The flask was evacuated and refilled with nitrogen 3 times. MeOH (330 mL, 0.1 M) was added in one portion and hydrogen gas was bubbled through the resulting solution for 1 min. The reaction mixture was stirred under hydrogen atmosphere at ambient temperature for 1 h after which it was filtered through a pad of celite. The reaction vessel and filter cake were washed with DCM (3×50 mL). The filtrate was concentrated under reduced pressure to afford compound S4 (5.63 g, 95%) as a yellow oil. The epimers were not separated nor characterized individually. Based on LCMS analysis, a 50:50 ratio of C16 epimers is obtained.

R_{f}=0.10 (10:1 DCM/MeOH, 1% NH₄OH). ¹H NMR (400 MHz, CDCl₃) δ (ppm) (epimers observed)=3.35-3.19 (m, 1H), 3.16-2.95 (m, 2H), 2.93-2.84 (m, 1H), 2.24-1.95 (m, 3H), 1.75-1.66 (m, 2H), 1.64-1.53 (m, 2H), 1.08-0.79 (m, 5H). ¹³C NMR (101 MHz, CDCl₃) δ (ppm) (epimers observed)=212.75, 210.44, 52.36, 51.28, 47.04, 46.74, 45.70, 45.41, 27.58, 26.71, 25.41, 24.49, 24.44, 24.23, 24.04, 23.52, 19.89, 19.73, 11.23, 10.86, 10.81, 10.67. HRMS (ESI)=m/z [M+H]⁺ calcd. For C₁₁H₁₈NO⁺, 180.1388; found, 180.1381.

### Octahydro-3,10-methanopyrido[1,2-a]azepin-9 (6H)-one (S3)

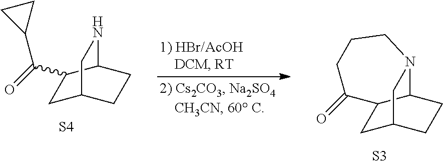

To a stirring solution of compound S4 (5.63 g, 31.42 mmol, 1.00 equiv.) in DCM (31 mL, 1.0 M) was added 33 wt % HBr/AcOH (16.27 mL, 94.27 mmol, 3.00 equiv.) in one portion. The solution was stirred at ambient temperature for 2 h after which it was concentrated under reduced pressure. The residue was dried under high vacuum for 1 h and then stirred vigorously in diethyl ether (50 mL). The diethyl ether was decanted, and the residue was dried under high vacuum. The alkyl bromide (S1 and S2) was isolated as a 1:1 mixture of diastereomers as an orange foam (10.71 g, 100% yield) and characterized by ¹H NMR. ¹H NMR (400 MHz, CDCl₃) δ (ppm)=9.77 (b, 1H), 9.26 (b, 2H), 7.71 (b, 1H), 4.01 (s, 1H), 3.93 (s, 1H), 3.62-3.24 (m, 8H), 2.92-2.41 (m, 6H), 2.27-2.00 (m, 8H), 1.98-1.85 (m, 2H), 1.79-1.54 (m, 8H).

A flask was charged with alkyl bromide diastereomers S1 and S2 (10.71 g, 31.41 mmol, 1.00 equiv.), anhydrous Cs₂CO₃ (15.35 g, 47.13 mmol, 1.50 equiv.), and anhydrous Na₂SO₄ (13.38 g, 94.26 mmol, 3.00 equiv.). The flask was evacuated and refilled with nitrogen 3 times after which anhydrous CH₃CN (314 mL, 0.1 M) was added in one portion. The reaction mixture was heated to 60° C., and stirred for 6 h. The heterogeneous mixture was cooled to ambient temperature and filtered. The reaction vessel and filter cake were washed with DCM. The filtrate was concentrated under reduced pressure and the residue was purified via chromatography on silica gel (20:1 DCM/MeOH) to afford compound S3 (2.31 g, 41% over 2 steps) as a light brown oil. R_{f}=0.40 (10:1 DCM/MeOH). ¹H NMR (400 MHz, CDCl₃) δ (ppm)=3.20-3.17 (m, 1H), 3.10-2.98 (m, 2H), 2.92 (dd, J=9.6, 4.1 Hz, 1H), 2.88-2.81 (m, 1H), 2.59 (d, J=9.7 Hz, 1H), 2.54 (dd, J=12.5, 6.8 Hz, 1H), 2.49 (d, J=12.5 Hz, 1H), 2.07-1.93 (m, 3H), 1.93-1.84 (m, 1H), 1.80-1.76 (m, 1H), 1.74-1.65 (m, 1H), 1.61-1.47 (m, 3H). ¹³C NMR (101 MHz, CDCl₃) δ (ppm)=216.49, 54.40, 51.61, 50.56, 50.37, 43.50, 29.56, 28.47, 25.82, 23.74, 20.80. HRMS (ESI)=m/z [M+H]⁺ calcd. For C₁₁H₁₈NO⁺, 180.1388; found, 180.1384.

### Example 10: Desethylibogaine (30)

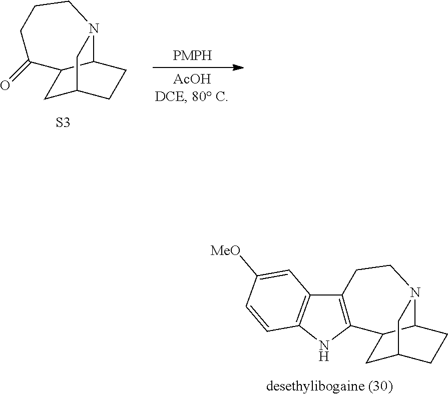

A flask was charged with compound S3 (1.25 g, 6.97 mmol, 1.00 equiv.) and para-methoxy phenylhydrazine. HCl (1.82 g, 10.45 mmol, 1.50 equiv.). The flask was evacuated and refilled with nitrogen 3 times after which anhydrous DCE (69.70 mL, 0.10 M) and AcOH (5.98 mL, 104.59 mmol, 15.00 equiv.) was added. The resulting mixture was degassed by bubbling nitrogen through the solution for 10 min. The flask was heated to 80° C., and stirred for 12 h after which it was cooled to ambient temperature and diluted with DCM (30 mL). Saturated aqueous NaHCO₃ was added to the reaction mixture until the pH was adjusted to 7-8. The organic layer was separated, and the aqueous layer was further extracted with DCM (2×50 mL). The combined organic extracts were dried over sodium sulfate, filtered and concentrated under reduced pressure. Purification via chromatography on silica gel (gradient elution 20:1→10:1 DCM/MeOH, 0.25% NH₄OH) afforded desethylibogaine 30 (1.65 g, 84%) as a yellow foam. R_{f}=0.38 (10:1 DCM/MeOH). ¹H NMR (400 MHz, CDCl₃) δ (ppm)=7.61 (s, 1H), 7.14 (d, J=8.6 Hz, 1H), 6.93 (s, 1H), 6.77 (d, J=9.9 Hz, 1H), 3.86 (s, 3H), 3.42-3.33 (m, 1H), 3.32-3.19 (m, 3H), 3.17-3.09 (m, 2H), 2.98 (dd, J=11.8, 4.7 Hz, 1H), 2.73-2.65 (m, 1H), 2.19-2.08 (m, 2H), 1.95-1.88 (m, 1H), 1.82-1.57 (m, 4H). ¹³C NMR (101 MHz, CDCl₃) δ (ppm)=154.22, 142.34, 129.90, 129.75, 111.17, 111.13, 109.05, 100.33, 56.15, 54.48, 54.25, 50.18, 39.23, 34.58, 29.16, 25.32, 23.41, 20.05. HRMS (ESI)=m/z [M+H]⁺ calcd. For C₁₈H₂₃N₂O⁺, 283.1808; found, 283.1815. IR (Smart iTX Diamond)=v 3392, 3048, 2927, 2834, 1623, 1587, 1515, 1484, 1364, 731 cm⁻¹.

### Example 11: Desethyl-10-fluoroibogamine (31)

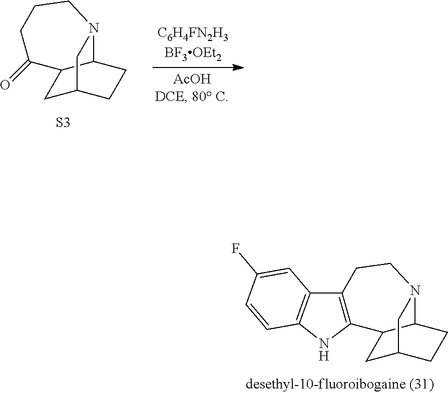

A flask was charged with compound S3 (1.10 g, 6.13 mmol, 1.00 equiv.) and para-fluorophenylhydrazine. HCl (1.49 g, 9.20 mmol, 1.50 equiv.). The flask was evacuated and refilled with nitrogen 3 times after which anhydrous DCE (61.30 mL, 0.1 M) and AcOH (5.25 mL, 91.95 mmol, 15.00 equiv.) was added. The resulting mixture was degassed by bubbling nitrogen through the solution for 10 min. The flask was heated to 80° C., and stirred for 1 h after which it was cooled to ambient temperature and BF₃·OEt₂ (0.907 mL, 7.35 mmol, 1.20 equiv.) was added in one portion. The resulting mixture was heated at 80° C. for 12 h after which it was cooled to ambient temperature and diluted with DCM (100 mL). Saturated aqueous NaHCO₃ was added to the reaction mixture until the pH was adjusted to 7-8. The organic layer was separated, and the aqueous layer was further extracted with DCM (2×50 mL). The combined organic fractions were dried over sodium sulfate, filtered, and concentrated under reduced pressure. Purification via chromatography on silica gel (gradient elution 20:1→10:1 DCM/MeOH, 0.25% NH₄OH) afforded desethyl-10-fluoroibogamine (1.33 g, 80%) as an orange foam. R_{f}=0.44 (10:1 DCM/MeOH). ¹H NMR (400 MHz, CDCl₃) δ (ppm)=7.84 (s, 1H), 7.14 (dd, J=8.7, 4.4 Hz, 1H), 7.09 (dd, J=9.8, 2.5 Hz, 1H), 6.83 (ddd, J=9.3, 8.7, 2.5 Hz, 1H), 3.39-3.22 (m, 3H), 3.13 (d, J=2.5 Hz, 2H), 3.04 (dt, J=3.7, 1.8 Hz, 1H), 2.96 (ddd, J=11.6, 4.7, 1.7 Hz, 1H), 2.62-2.50 (m, 1H), 2.02 (ddt, J=13.4, 9.9, 4.8 Hz, 2H), 1.89 (p, J=2.9 Hz, 1H), 1.80-1.62 (m, 4H). ¹³C NMR (101 MHz, CDCl₃) δ (ppm)=157.88 (¹J_{CF}=233.7 Hz), 143.87, 130.96, 130.09 (¹J_{CF}=9.3 Hz), 110.68 (¹J_{CF}=9.7 Hz), 109.67, 108.93 (¹J_{CF}=26.2 Hz), 102.91 (¹J_{CF}=23.4 Hz), 54.04, 53.76, 50.03, 40.04 (d, J=3.4 Hz), 34.70, 29.88, 25.54, 23.64, 20.18. HRMS (ESI)=m/z [M+H]⁺ calcd. For C₁₇H₂₀FN₂⁺, 271.1608; found, 271.1609. IR (Smart iTX Diamond)=v 3164, 3048, 2939, 2873, 1701, 1654, 1583, 1451, 1390, 732 cm⁻¹.

### Example 12: Desethyl-10-Chloroibogamine Fumarate Salt

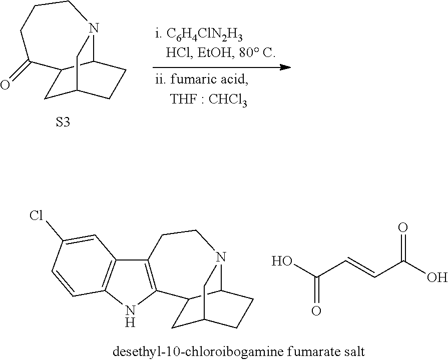

A vial was charged with compound S3 (60.25 mg, 0.336 mmol, 1 equiv.) and p-chlorophenylhydrazine·HCl (179.05 mg, 1 mmol, 3 equiv.). The starting materials were dissolved in EtOH (3.4 mL) and conc. HCl (0.112 mL, 1.34 mmol, 4 equiv.) was then added. The resulting mixture was stirred and heated to 80° C. for 24 h. The reaction was concentrated under reduced pressure, then taken up in DCM (5 mL) and basified with IM NaOH (2 mL). The mixture was diluted with DI H₂O (5 mL). The layers were separated, and the organic layer was collected. The aqueous layer was then extracted with DCM (7×5 mL). The organic layers were combined and dried over Na₂SO₄. The solution was filtered, and the filtrate was concentrated under reduced pressure. The crude oily material was then purified by column chromatography on silica gel using a gradient solvent system (10:1 DCM:MeOH to 10:1 DCM:MeOH+0.5% NH₄OH to 10:1 DCM:MeOH+1% NH₄OH) and the isolated material was used in the salting step without further purification or characterization. R_{f}=0.29 (in 10:1 DCM:MeOH+1% NH₄OH).

A 5-mL vial was charged with desethyl-10-chloroibogamine (84.4 mg, 0.294 mmol, 1 equiv.) and dissolved in CHCl₃ (1 mL). A second vial was charged with fumaric acid (27.33 mg, 0.235 mmol, 0.8 equiv.) and dissolved in THF (1 mL). The solution of desethyl-10-chloroibogamine in CHCl₃ was added dropwise to the solution of fumaric acid in THF. The mixture was allowed to cool at −20° C. for 18 h, before it was filtered and the filter cake washed with cold hexanes, then cold acetone. The product was obtained as the 1:1 desethyl-10-chloroibogamine: fumaric acid salt and was a light-yellow powder (55 mg, 47%). ¹H NMR (400 MHz, MeOD) δ 7.42 (d, J=2.0 Hz, 1H), 7.22 (d, J=8.6 Hz, 1H), 7.02 (dd, J=8.6, 2.0 Hz, 1H), 6.68 (s, 2H, fumaric acid), 3.51 (ddd, J=15.9, 9.8, 3.5 Hz, 3H), 3.41 (d, J=2.5 Hz, 2H), 3.34 (d, J=4.9 Hz, 1H), 3.29-3.25 (m, 1H), 3.04 (dt, J=17.5, 3.7 Hz, 1H), 2.33 (t, J=12.8 Hz, 1H), 2.18 (ddd, J=17.1, 9.3, 4.1 Hz, 1H), 2.14-2.07 (m, 1H), 2.01-1.91 (m, 1H), 1.90-1.71 (m, 3H). HRMS (ESI)=m/z [M+H]⁺ calcd. for C₁₇H₂₀ClN₂, 287.1310; found, 287.1302.

### Example 13: Desethyl-10,11-methylenedioxyibogamine Fumarate Salt

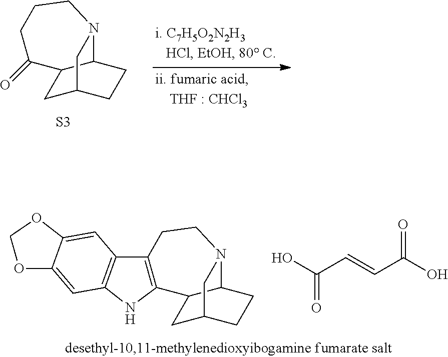

A flask was charged with compound S3 (60.25 mg, 0.336 mmol, 1 equiv.) and 3,4-methylenedioxyphenylhydrazine·HCl (188.61 mg, 1 mmol, 3 equiv.). The starting materials were dissolved in EtOH (3.4 mL) and conc. HCl (0.112 mL) was then added. The resulting mixture was stirred and heated to 80° C. for 24 h. The reaction was concentrated under reduced pressure, then taken up in DCM (5 mL) and basified with 1 M NaOH (2 mL). The mixture was diluted with DI H₂O (5 mL). The layers were separated, and the organic layer was collected. The aqueous layer was then extracted with DCM (7×5 mL). The organic layers were combined and dried over Na₂SO₄. The solution was then filtered, and the filtrate was concentrated under reduced pressure. The crude oily material was then purified by column chromatography on silica gel using a gradient solvent system (10:1 DCM:MeOH to 10:1 DCM:MeOH+0.5% NH₄OH to 10:1 DCM:MeOH+1% NH₄OH) and the isolated material was used in the salting step without further purification or characterization. (R_{f}=0.21 (in 10:1 DCM:MeOH+1% NH₄OH).

A vial was charged with desethyl-10,11-methylenedioxyibogamine (97.2 mg, 0.33 mmol, 1 equiv.) and dissolved in CHCl₃ (1 mL). A second vial was charged with fumaric acid (30.45 mg, 0.2624 mmol, 0.8 equiv.) and the powder was subsequently dissolved in THF (1 mL). The solution of desethyl-10,11-methylenedioxyibogamine in CHCl₃ was added dropwise to the solution of fumaric acid in THF. The mixture was allowed to cool at −20° C. for 18 h, before the mixture was filtered and the filter cake washed with cold hexanes and acetone. The product was isolated as the 2:1 desethyl-10,11-methylenedioxyibogamine:fumaric acid salt and was a light-brown powder (74.4 mg, 64%). ¹H NMR (400 MHz, DMSO) δ (ppm) 10.46 (s, 1H), 6.87 (s, 1H), 6.76 (s, 1H, fumaric acid), 6.53 (s, 1H), 5.87 (s, 2H), 3.27-2.94 (m, 8H), 2.70-2.53 (m, 1H), 2.08 (t, J=12.5 Hz, 1H), 2.01-1.80 (m, 2H), 1.75-1.50 (m, 3H). HRMS (ESI)=m/z [M+H]⁺ calcd. for C₁₈H₂₁N₂O₂⁺, 297.1598; found, 297.1598.

### Phenyl pyridine-1(2H)-carboxylate (S14)

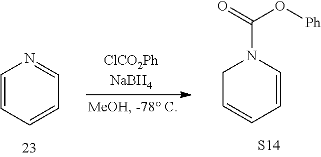

A flask was charged with methanol (200 mL, 0.5 M) and pyridine (8.05 mL, 100 mmol, 1.00 equiv.) and cooled to −78° C. after which sodium borohydride (4.54 g, 120 mL, 120 mmol) was added in one portion. Phenyl chloroformate (15.05 mL, 120 mmol, 1.20 equiv.) was added dropwise over 1 h to the reaction mixture. The reaction mixture was stirred at −78° C. for an additional 3 h after which it was diluted in Et₂O (100 mL), poured into 1 M HCl (200 mL) and the layers were separated. The aqueous layer was extracted with Et₂O (2×100 mL) and the combined organic extracts were washed with 1 M NaOH (50 mL) followed by brine (50 mL). The organic extracts were dried over sodium sulfate, filtered, and concentrated under reduced pressure. The residue was recrystallized from ethanol to afford compound S14 (18.71 g, 93%) as a crystalline white solid. ¹H NMR (400 MHz, CDCl₃) δ (ppm) (rotamers observed): 7.39 (t, J=7.9 Hz, 2H), 7.28-7.21 (m, 1H), 7.20-7.12 (m, 2H), 6.95-6.79 (m, 1H), 5.91 (m, 1H), 5.66-5.53 (m, 1H), 5.33-5.21 (m, 1H), 4.60 (dd, J=4.1, 2.1 Hz, 1H), 4.47 (dd, J=4.1, 2.1 Hz, 1H). ¹³C NMR (101 MHz, CDCl₃) δ (ppm) (rotamers observed)=152.46, 151.43, 150.88, 150.70 (d, J=10.0 Hz), 129.29, 125.99, 125.61 (d, J=4.5 Hz), 125.29, 122.16, 121.77, 121.48, 119.39, 118.84, 105.94, 105.75, 44.21, 43.73. HRMS (ESI)=m/z [M+H]⁺ calcd. For C₁₂H₁₂NO₂⁺, 202.0868; found, 202.0866.

### (S)-3-acryloyl-4-benzyloxazolidin-2-one (S15)

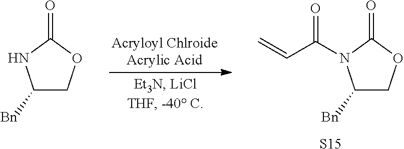 [SMILES: O=C1N[C@@H](Cc2ccccc2)CO1 ; C=CC(=O)N1C(=O)OC[C@@H]1Cc1ccccc1 ; "S15"]

A flask was sequentially charged with THF (250 mL, 0.20 M), acrylic acid (4.44 mL, 64.77 mmol, 1.30 equiv.) and triethylamine (17.36 mL, 124.57 mmol, 2.50 equiv.) under nitrogen atmosphere and cooled to −40° C. using a dry ice/acetonitrile cooling bath. Acryloyl chloride (4.83 mL, 59.78 mmol, 1.20 equiv.) was added dropwise to the reaction mixture and the resulting milky yellow solution was stirred at −40° C. for 1 h. Lithium chloride (2.64 g, 62.27 mmol, 1.25 equiv.) followed by(S)-4-benzyloxazolidin-2-one (8.83 g, 49.82 mmol, 1.00 equiv.) was added in one portion to the reaction mixture and the solution was slowly warmed to ambient temperature over the course of 24 h. The reaction mixture was then concentrated under reduced pressure and the resulting residue was dissolved in DCM (200 mL) and poured into a solution of 2 M HCl (100 mL). The layers were separated, and the aqueous layer was washed with DCM (2×100 mL). The organic extracts were combined, dried over sodium sulfate, filtered, and concentrated under reduced pressure. The residue was purified via chromatography on silica gel (gradient elution hexanes→7:3 hexanes/EtOAc) to afford compound S15 (7.83 g, 68%) as a white solid. R_{f}=0.6 (7:3 hexanes/EtOAc). ¹H NMR (400 MHz, CDCl₃) δ (ppm)=7.51 (dd, J=17.0, 10.4 Hz, 1H), 7.40-7.17 (m, 5H), 6.61 (dd, J=17.0, 1.8 Hz, 1H), 5.94 (dd, J=10.4, 1.8 Hz, 1H), 4.74 (ddt, J=9.5, 7.0, 3.4 Hz, 1H), 4.30-4.14 (m, 2H), 3.35 (dd, J=13.4, 3.3 Hz, 1H), 2.81 (dd, J=13.4, 9.5 Hz, 1H). ¹³C NMR (101 MHz, CDCl₃) δ (ppm)=164.91, 153.34, 135.23, 131.94, 129.46, 129.01, 127.41, 127.38, 66.29, 55.32, 37.82. HRMS (ESI)=m/z [M+H]⁺ calcd. For C₁₃H₁₄NO₃⁺, 232.0978; found, 232.0971.

### Phenyl (1R,4R,7R)-7-((S)-4-benzyl-2-oxooxazolidine-3-carbonyl)-2-azabicyclo[2.2.2]oct-5-ene-2-carboxylate (S16)

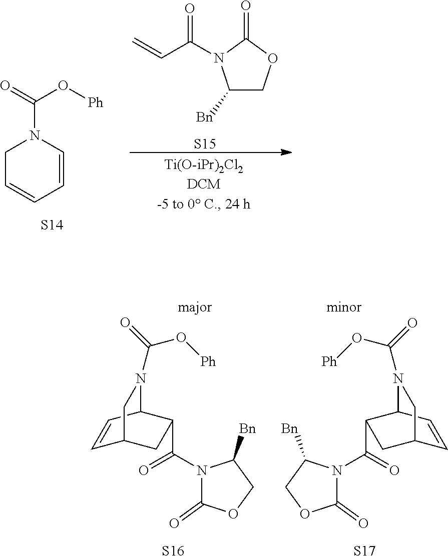

A flask was sequentially charged with activated 4 Å molecular sieves (16.00 g), compound S15 (6.50 g, 28.10 mmol, 1.00 equiv.), and Ti(O-iPr)₂Cl₂ (11.78 g, 49.73 mmol, 1.77 equiv.). The flask was evacuated and refilled with nitrogen 3 times after which DCM (175 mL) was added and the resulting milky solution was stirred at ambient temperature for 1 h. The flask was then cooled to 0° C. in an ice bath and a solution of compound S14 (11.31 mmol, 56.20 mmol, 2.00 equiv.) in DCM (175 mL) was added dropwise over 30 min. The reaction mixture was stirred at −10° C. to 0° C. for 48 h after which it was quenched with saturated aqueous NaHCO₃ (200 mL). The organic layer was separated, and the aqueous layer was washed with DCM (2×200 mL). The combined organic extracts were dried over sodium sulfate, filtered, and concentrated under reduced pressure. The reside was purified via chromatography on silica gel (gradient elution 10:1→7:3 hexanes/EtOAc) to afford compound S16 and S17 (11.91 g, 98%, 77:23) as a white foam. The diastereomeric ratio of the unpurified residue is 77:23 (¹H NMR) and major diastereomer S16 (9.17 g, 75%) can be separated via chromatography. The exact structure of diastereomer S16 was assigned retroactively after completing the synthesis of (+)-ibogaine and comparing its chiral LCMS spectra with that of racemic ibogaine and natural (−)-ibogaine.

Major Isomer (S16). R_{f}=0.25 (7:3 hexanes/EtOAc). ¹H NMR (400 MHz, MeOD) δ (ppm) (rotamers observed)=7.46-7.10 (m, 10H), 6.65-6.43 (m, 2H), 5.28 (ddd, J=6.1, 2.8, 1.3 Hz, 0.5H), 5.12 (ddd, J=5.8, 2.7, 1.5 Hz, 0.5H), 4.73-4.58 (m, 1H), 4.34-4.13 (m, 3H), 3.59 (dd, J=10.3, 2.0 Hz, 0.5H), 3.38 (dd, J=10.5, 2.0 Hz, 0.5H), 3.28-3.20 (m, 0.5H), 3.14 (dd, J=13.5, 3.3 Hz, 1H), 3.07-3.01 (m, 0.5H), 2.92 (dtd, J=15.0, 7.8, 4.1 Hz, 2H), 2.24-2.14 (m, 0.5H), 2.10-1.99 (m, 0.5H), 1.97-1.87 (m, 0.5H), 1.82-1.71 (m, 0.5H). ¹³C NMR (101 MHz, MeOD) δ (ppm) (rotamers observed)=173.93, 173.53, 155.26, 154.85, 154.71, 152.72, 152.68, 137.01, 136.95, 136.69, 135.83, 132.05, 130.84, 130.59, 130.57, 130.31, 130.29, 129.83, 129.78, 128.20, 126.46, 123.02, 122.92, 67.88, 67.81, 56.64, 56.56, 45.81, 45.39, 38.48, 38.33, 32.20, 31.95, 28.34, 27.44. HRMS (ESI)=m/z [M+H]⁺ calcd. For C₂₅H₂₅N₂O₅⁺, 433.1768; found, 433.1760.

### (1R,4R,6R)-2-(phenoxycarbonyl)-2-azabicyclo[2.2.2]oct-7-ene-6-carboxylic Acid (S18)

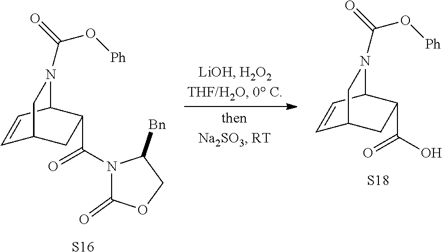

A flask was sequentially charged with compound S16 (8.50 g, 19.65 mmol, 1.00 equiv.), THF (260 mL) and 30% aqueous H₂O₂ (9.23 mL, 90.39 mmol, 4.60 equiv.) and cooled to 0° C. in an ice bath. A solution of LiOH (753 mg, 31.44 mmol, 1.60 equiv.) in H₂O (130 mL) was added via syringe pump (30 mL/hr) to the reaction and the resulting solution was slowly warmed to ambient temperature over 5 h. A solution of Na₂SO₃ (12.35 g, 98.05 mmol, 4.98 equiv.) in H₂O (50 mL) was added and the reaction mixture was stirred at ambient temperature for 1 h. A 5 M solution of aqueous NaOH (75 mL) was added and the resulting mixture was poured into DCM (100 mL) and the layers were separated. The aqueous layer was washed with DCM (2×50 mL) and then acidified to pH 3 with 4 M HCl (100 mL). The acidic aqueous layer was then extracted with DCM (3×150 mL) and the organic extracts were combined, dried over sodium sulfate, filtered, and concentrated under reduced pressure to afford compound S18 (5.21 g, 97%) as a white solid. ¹H NMR (400 MHz, MeOD) δ (ppm) (rotamers observed): 7.44-7.30 (m, 2H), 7.27-7.19 (m, 1H), 7.18-7.07 (m, 2H), 6.55 (ddd, J=8.2, 6.6, 1.5 Hz, 1H), 6.43 (tdd, J=8.0, 5.9, 1.5 Hz, 1H), 5.24 (ddd, J=5.9, 3.3, 1.3 Hz, 0.5H), 5.11 (ddd, J=5.9, 3.3, 1.4 Hz, 0.5H), 3.54 (dd, J=10.3, 2.2 Hz, 1H), 3.29-3.10 (m, 2H), 3.09-2.88 (m, 2H), 2.06-1.80 (m, 2H). ¹³C NMR (101 MHz, MeOD) δ (ppm) (rotamers observed)=175.85, 155.52, 154.90, 152.68, 137.08, 137.00, 131.23, 131.10, 130.39, 130.33, 126.55, 126.51, 122.89, 44.89, 44.59, 32.09, 31.80, 26.95, 26.83. HRMS (ESI)=m/z [M+H]⁺ calcd. For C₁₅H₁₆NO₄⁺, 274.1078; found, 274.1071. Specific Rotation=[α]_{D}²⁰=8.4 (c=0.15 in MeOH).

### Phenyl (1R,4R,7R)-7-(methoxy(methyl) carbamoyl)-2-azabicyclo[2.2.2]oct-5-ene-2-carboxylate (S23)

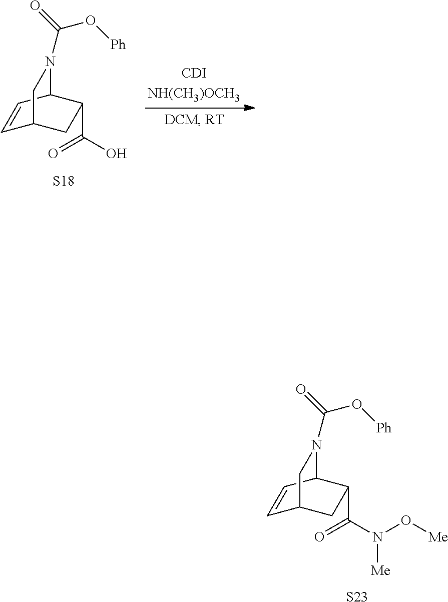 [SMILES: O=C(O)C1CC2C=CC1N(C(=O)Oc1ccccc1)C2 ; "S18" ; CON(C)C(=O)C1CC2C=CC1N(C(=O)Oc1ccccc1)C2 ; "S23"]

To a stirring solution of compound S18 (4.85 g, 17.74 mmol, 1.00 equiv.) in DCM (88 mL, 0.20 M) was added 1,1′-carbonyldiimidazole (CDI) (3.74 g, 23.06 mmol, 1.30 equiv.) in one portion. The resulting clear solution was stirred at ambient temperature for 1 h after which N,O-dimethylhydroxylamine hydrochloride (3.46 g, 35.48 mmol, 2.00 equiv.) was added in one portion. The mixture was stirred at ambient temperature for 12 h after which 4 M HCl (100 mL) was added, and the layers were separated. The aqueous layer was further extracted with DCM (2×100 mL) and the combined organic extracts were dried over sodium sulfate, filtered and concentrated under reduced pressure to afford compound S23 (5.55 g, 99%) as a white solid. ¹H NMR (400 MHz, CDCl₃) δ (ppm) (rotamers observed): 7.32 (ddd, J=8.2, 7.2, 2.3 Hz, 2H), 7.19-7.07 (m, 3H), 6.54-6.38 (m, 2H), 5.20-5.03 (m, 1H), 3.70 (d, J=4.4 Hz, 3H), 3.53-3.32 (m, 2H), 3.22-3.03 (m, 4H), 2.87 (dq, J=5.1, 2.6 Hz, 1H), 1.97 (dddd, J=12.5, 9.6, 5.6, 2.6 Hz, 1H), 1.77 (dddd, J=12.3, 9.3, 4.7, 2.9 Hz, 1H). ¹³C NMR (101 MHz, CDCl₃) δ (ppm) (rotamers observed)=153.50, 152.92, 151.34, 134.66, 134.18, 130.96, 130.50, 129.28, 129.23, 125.19, 121.74, 121.63, 61.41, 61.30, 47.76, 47.36, 47.32, 47.05, 42.38, 41.82, 30.87, 30.60, 27.56, 27.34. HRMS (ESI)=m/z [M+H]⁺ calcd. For C₁₇H₂₁N₂O₄+, 317.1498; found, 317.1799. Specific Rotation=[α]_{D}²⁰=18.4 (c=0.10 in MeOH).

### Phenyl (1R,4R,7R)-7-(cyclopropanecarbonyl)-2-azabicyclo[2.2.2]oct-5-ene-2-carboxylate (S19)

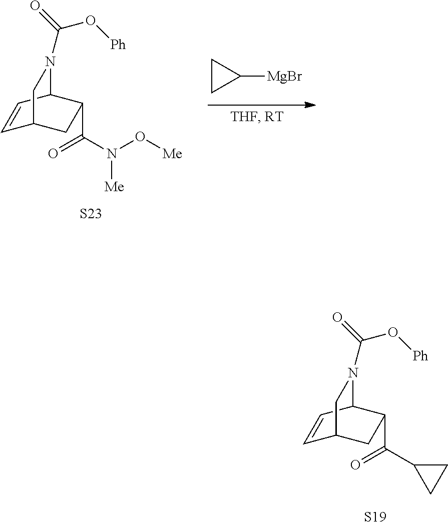

A flask was charged with compound S23 (5.55 g, 17.56 mmol, 1.00 equiv.) after which THF (175 mL, 0.1 M) was added under a stream of nitrogen. A solution of 1.0 M cyclopropyl magnesium bromide (52.68 mL, 52.68 mmol, 3.00 equiv.) was added dropwise over 2 h to the reaction mixture at ambient temperature. The resulting solution was stirred at ambient temperature for 1 h after which it was quenched with saturated aqueous NH₄Cl (100 mL). The mixture was diluted in DCM (200 mL) and poured into water (100 mL). The layers were separated, and the aqueous layer was further washed with DCM (2×100 mL). The organic fractions were collected, dried over sodium sulfate, filtered, and concentrated under reduced pressure. The resulting residue was purified via chromatography on silica gel (7:3 hexanes/EtOAc) to afford compound S19 (5.01 g, 96%) as a white solid. R_{f}=0.45 (7:3 hexanes/EtOAc). ¹H NMR (400 MHz, CDCl₃) δ (ppm) (rotamers observed): 7.36 (tt, J=6.9, 2.1 Hz, 2H), 7.19 (td, J=7.2, 1.3 Hz, 1H), 7.16-7.09 (m, 2H), 6.50-6.27 (m, 2H), 5.32 (dq, J=3.0, 1.3 Hz, 1H), 3.56-3.36 (m, 2H), 3.23-3.06 (m, 1H), 2.92 (s, 1H), 2.10-1.77 (m, 3H), 1.08-0.84 (m, 4H). ¹³C NMR (101 MHz, CDCl₃) δ (ppm) (rotamers observed)=208.72, 208.37, 151.33, 135.03, 130.13, 129.29, 129.25, 125.28, 121.73, 52.69, 52.17, 47.67, 47.17, 30.83, 30.54, 25.12, 24.32, 19.45, 19.34, 11.52, 11.39, 10.97, 10.71. HRMS (ESI)=m/z [M+H]⁺ calcd. For C₁₈H₂₀NO₃⁺, 298.1438; found, 298.1433. Specific Rotation=[α]_{D}²⁰=12.5 (c=0.15 in MeOH). Enantomeric Excess (ee)=97.31%.

### Benzyl (1S,4S)-7-(cyclopropanecarbonyl)-2-azabicyclo[2.2.2]oct-5-ene-2-carboxylate (20)

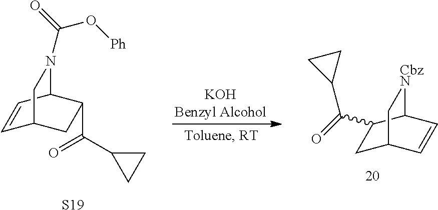 [SMILES: O=C(C1CC1)C1CC2C=CC1N(C(=O)Oc1ccccc1)C2 ; O=C(C1CC1)C1CC2C=CC1N(C(=O)OCc1ccccc1)C2 ; "S19"]

A flask was sequentially charged with compound S19 (5.00 g, 16.81 mmol, 1.00 equiv.), toluene (84 mL, 0.2 M) and benzyl alcohol (2.09 mL, 20.17 mmol, 1.20 equiv.). Powdered KOH (1.13 g, 20.17, 1.20 equiv.) was added to the reaction mixture in one portion and the resulting solution was stirred at ambient temperature for 12 h. The reaction was diluted in DCM (100 mL), poured into water (50 mL) and the layers were separated. The aqueous layer was washed with DCM (2×50 mL) and the combined organic extracts were dried over sodium sulfate, filtered, and concentrated under reduced pressure. The resulting residue was purified via chromatography on silica gel (7:3 hexanes/EtOAc) to afford compound 20 (4.92 g, 94%) as a light-yellow oil. Spectral data for compound 20 is in accordance with a previously noted procedure. Compound 20 was isolated as a 50:50 ratio of C16 epimers.

### C. Biological Examples

### Example A: Assays

Data Analysis and Statistics. Treatments were randomized, and data were analyzed by experimenters blinded to treatment conditions. Data are represented as mean±SEM, unless otherwise noted, with asterisks indicating *p<0.05. **p<0.01. ***p<0.001, and ****p<0.0001.

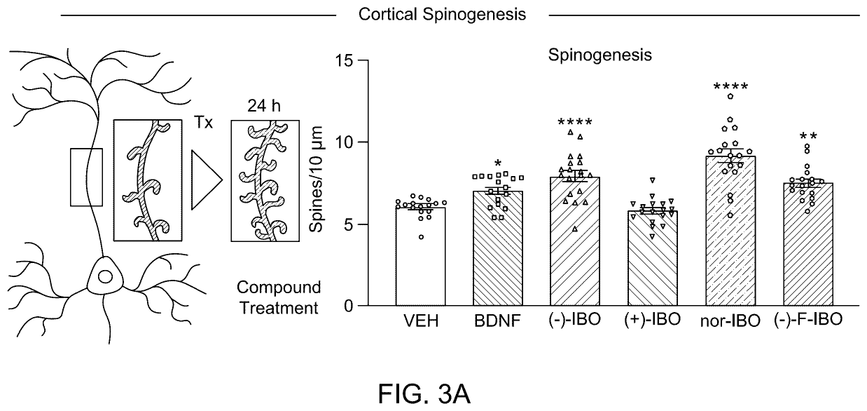

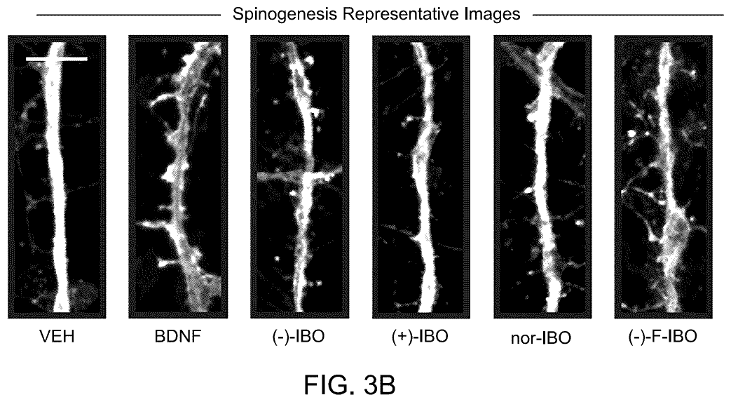

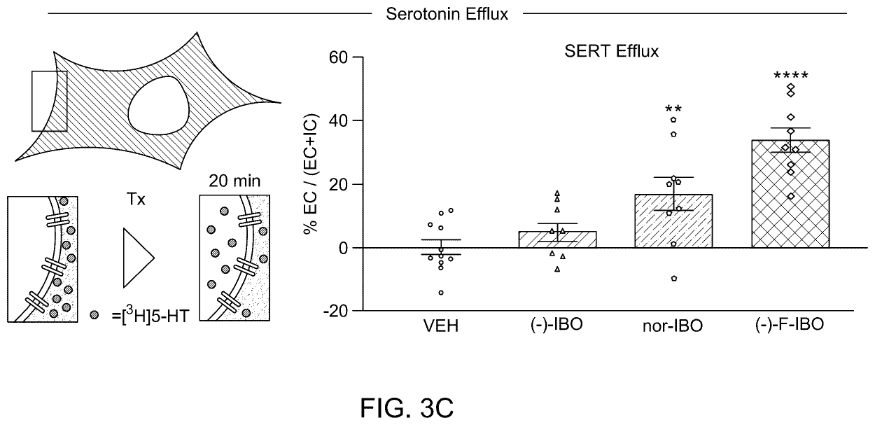

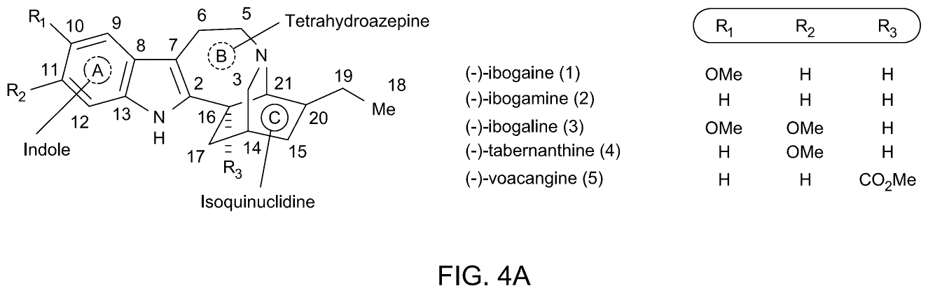

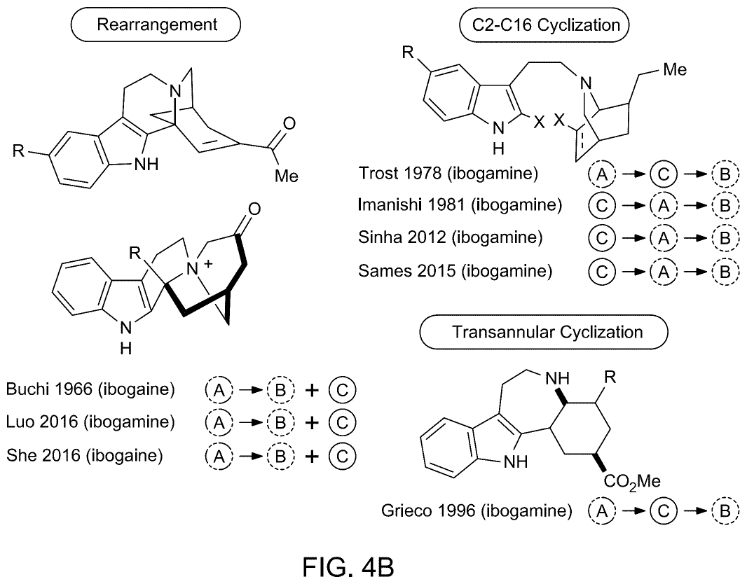

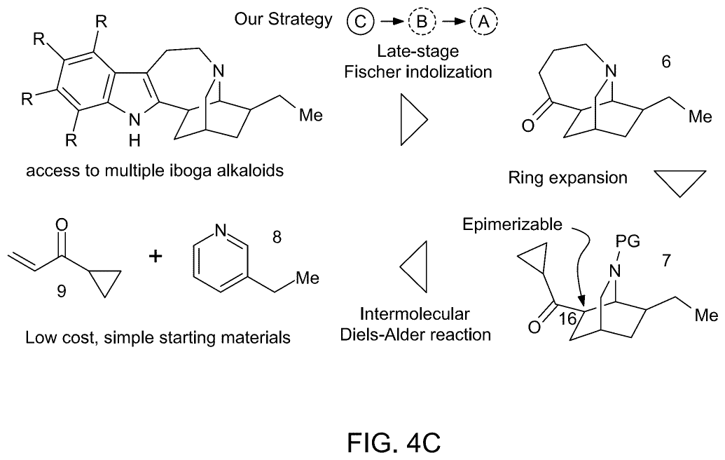

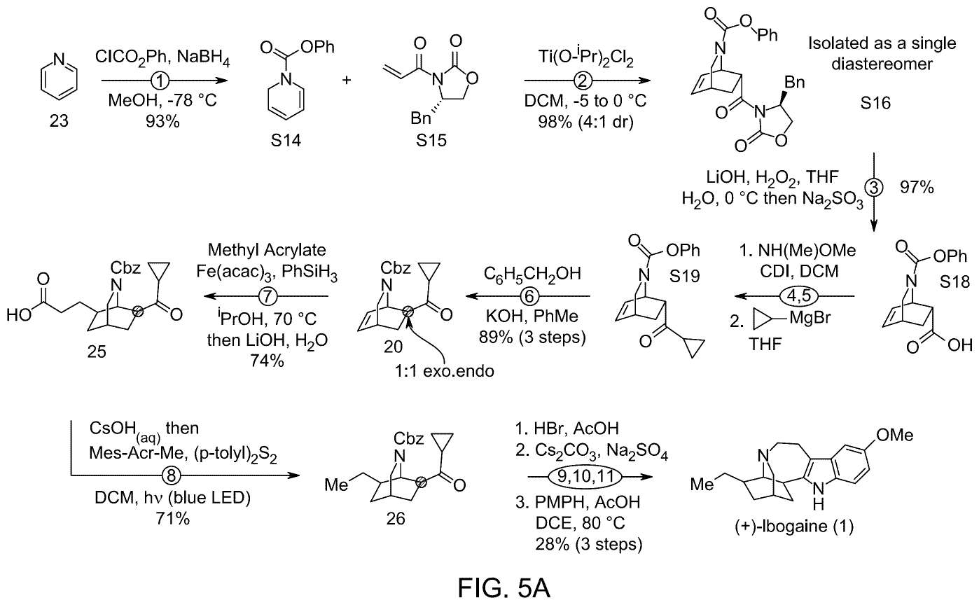

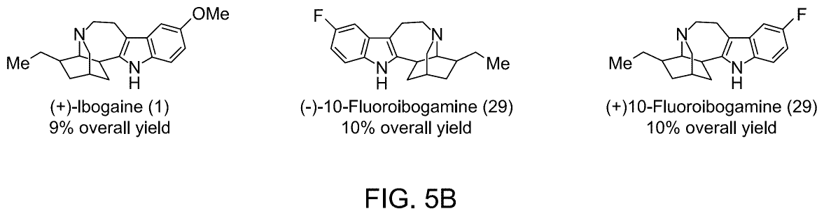

Spinogenesis Experiments. Spinogenesis assays were performed according to previously described protocols using cultured embryonic rat cortical neurons. Neurons were treated on DIV20 and fixed 24 h later (DIV21). An anti-chicken MAP2 antibody (1:10,000; EnCor, CPCA-MAP2) was used to visualize dendrites. Imaging was performed using a Nikon HCA Confocal microscope with 100×/NA 1.45 oil objective. The vehicle and positive controls were DMSO (0.1% in media) and BDNF (50 ng/ml), respectively. ImageJ (Version 2.9.0/1.53t) was used to measure the length of the dendrites. (see FIG. 3A and FIG. 3B).

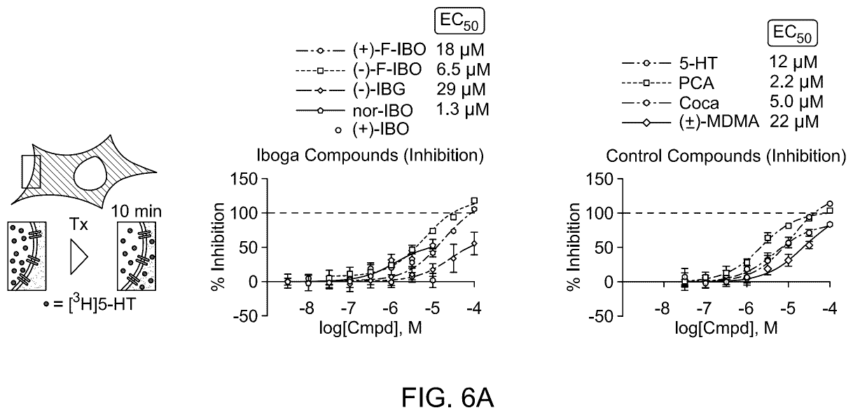

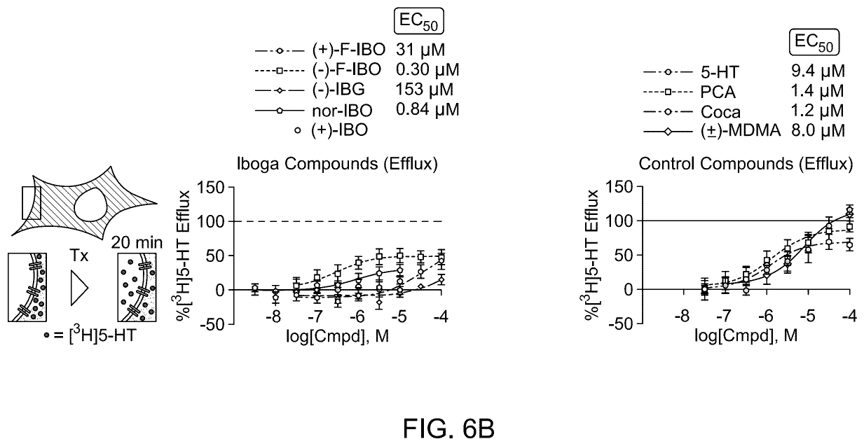

Serotonin Inhibition Experiments. Cells (HEK293T) were grown in Dulbecco's Modified Eagle Media (DMEM) supplemented with 10% fetal bovine serum (FBS). Plastic Costar 96-well plates were seeded at a density of 100,000 cells/well 24 h prior to the experiment and concurrently transfected using Lipofectamine™ 3000 Transfection Reagent according to the manufacturer's protocol. Cells were transfected with 0.1 μg of hSERT-N1-pEYFP (Addgene #70105) per well. Wells were washed (1×200 μL) with 1× Hank's Balanced Salt Solution supplemented with 2 mM MgCl₂ and 2 mM CaCl₂) (HBSS) as well as 5 mM HEPES and replenished with 100 μL of supplemented HBSS. The plate was placed in a 37° C. water bath for the reminder of the experiment and allowed to incubate for 30 min prior to beginning the experiment. The assay was initiated by adding a 100 μL mixture of 5-[1,2-³H(N)]-hydroxytryptamine creatinine sulfate ([³H]5-HT: #NET498001 MC, Lot: 3261835, Revvity) and respective drug onto a plate at final concentrations of 20 nM [³H]5-HT and various concentrations of drug (31.6 nM-100 μM). After 10 min, uptake was terminated by aspiration and wells washed with supplemented HBSS (3×200 μL). Cells were then lysed for 30 min by the addition of 30 μL of 10 mM NaOH, and 120 μL OptiPhase HiSafe (#1200.437; Revvity) was added to the cell lysates. Counts per minute (CPM) were quantified using MicroBeta2 microplate liquid scintillation counter. In GraphPad Prism, concentration responses were plotted as the difference of the VEH (0.1% DMSO) average for the plate and the count per minute (CPM) for each respective drug concentration (VEHavg−drug). The plots were normalized similarly, with the plate vehicle control (VEHavg−VEHavg) average being set to 0% inhibition and the difference between the VEH average and the positive control average (VEHavg−100 μM Cocaavg) set to 100% (see FIG. 6A). Outliers were removed by using the ROUT method (Q=10%) for each column separately.

Serotonin Efflux Experiments (24-well plates). Cells (HEK293T) were grown in Dulbecco's Modified Eagle Media (DMEM) supplemented with 10% fetal bovine serum (FBS). Plastic 24-well plates were seeded at a density of 200,000 cells/well 24 h prior to the experiment and concurrently transfected using Lipofectamine™ 3000 Transfection Reagent according to the manufacturer's protocol. Cells were transfected with 2 μg of hSERT-N1-pEYFP (Addgene #70105) per well. Wells were washed (1×500 μL) with 1× Hank's Balanced Salt Solution supplemented with 2 mM MgCl₂ and 2 mM CaCl₂) (HBSS) supplemented with 5 mM HEPES and replenished with 250 μL of supplemented HBSS. The plate was placed in a 37° C. water bath for the reminder of the experiment and allowed to incubate for 1 h prior to beginning the experiment. Next. 250 μL of HBSS containing 40 nM 5-[1,2-3H(N)]-hydroxytryptamine creatinine sulfate (NET498001 MC. Lot: 3147551) was added to the wells at a final concentration of 20 nM and incubated for 15 min to allow uptake into SERT-expressing cells. Uptake was terminated by aspiration of the wells followed by washing with supplemented HBSS (2×500 μL) and replenished with 400 μL of supplemented HBSS. Efflux was then initiated by adding 100 μL of drug (50 μM. 0.5% DMSO) solution in supplemented HBSS for 20 min at a final concentration of 10 μM (0.1% DMSO). Following the incubation. 250 μL media was collected from each well and placed into a scintillation vial containing 1 mL PerkinElmer Ultima Gold™ scintillation cocktail. Wells were washed with supplemented HBSS (3×500 μL) and cells were lysed for 30 min with addition of 250 μL of 10 mM NaOH. Cell lysates were collected in separate scintillation vials, as before, and served as the intracellular fraction. Sample activity was quantified with a Beckman LS 6000 liquid scintillation counter and recorded as disintegrations per minute (DPM). Bar graphs were reported as (extracellular)/(extracellular+intracellular) and normalized to the vehicle (0.1% DMSO) and positive (10 μM p-chloroamphetamine. PCA) controls as 0% and 100%, respectively (see FIG. 3C).

Serotonin Efflux Experiments (96-well plates, 31.6 nM-100 μM). Cells (HEK293T) were grown in Dulbecco's Modified Eagle Media (DMEM) supplemented with 10% fetal bovine serum (FBS). Plastic Costar 96-well plates were seeded at a density of 100,000 cells/well 24 h prior to the experiment and concurrently transfected using Lipofectamine™ 3000 Transfection Reagent according to the manufacturer's protocol. Cells were transfected with 0.1 μg of hSERT-N1-pEYFP (Addgene #70105) per well. Wells were washed (1×200 μL) with 1× Hank's Balanced Salt Solution supplemented with 2 mM MgCl₂ and 2 mM CaCl₂) (HBSS) as well as 5 mM HEPES and replenished with 100 μL of supplemented HBSS. The plate was placed in a 37° C. water bath for the reminder of the experiment and allowed to incubate for 30 min prior to beginning the experiment. Next. 100 μL of HBSS containing 80 nM 5-[1,2-3H(N)]-hydroxytryptamine creatinine sulfate ([³H]5-HT; #NET498001 MC. Lot: 3261835. Revvity) was added to the wells at a final concentration of 40 nM and incubated for 30 min to allow uptake into SERT-expressing cells. Uptake was terminated by aspiration of the wells followed by washing with supplemented HBSS (2×200 μL) and replenished with 180 μL of supplemented HBSS. Efflux was then initiated by adding 20 μL of drug (316 nM-1 mM. 1% DMSO) solution in supplemented HBSS for 20 min at final concentrations ranging from 31.6 nM-100 μM (0.1% DMSO). Following the incubation, wells were washed with supplemented HBSS (3×200 μL). Cells were then lysed for 30 min by the addition of 30 μL of 10 mM NaOH, and 120 μL OptiPhase HiSafe (#1200.437; Revvity) was added to the cell lysates. Counts per minute (CPM) were quantified using MicroBeta2 microplate liquid scintillation counter. In GraphPad Prism, concentration responses were plotted as the difference of the VEH (0.1% DMSO) average for the plate and the count per minute (CPM) for each respective drug concentration (VEHavg-drug). The plots were normalized similarly, with the plate vehicle control (VEHavg-VEHavg) average being set to 0% efflux and the difference between the VEH average and the positive control average (VEHavg−100 μM PCAavg) set to 100% (see FIG. 6B). Outliers were removed by using the ROUT method (Q=10%) for each column separately.

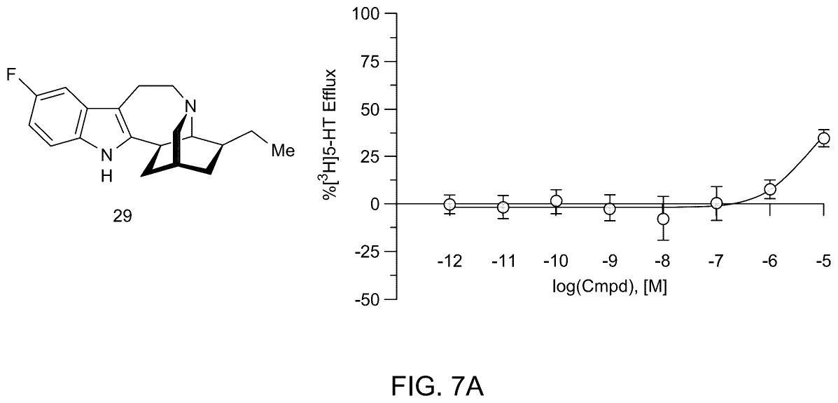

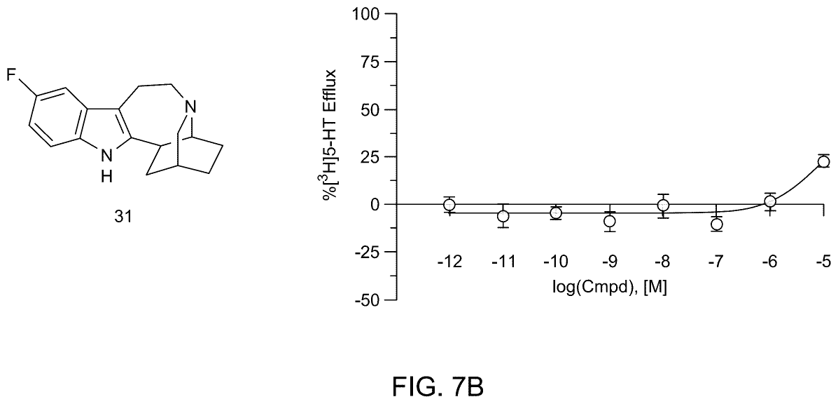

Serotonin Efflux Experiments (96-well plates, 1 nM-10 μM). Cells (HEK293T) were grown in Dulbecco's Modified Eagle Media (DMEM) supplemented with 10% fetal bovine serum (FBS). Plastic Costar 96-well plates were seeded at a density of 100,000 cells/well 24 h prior to the experiment and concurrently transfected using Lipofectamine™ 3000 Transfection Reagent according to the manufacturer's protocol. Cells were transfected with 0.1 μg of hSERT-N₁-pEYFP (Addgene #70105) per well. Wells were washed (1×200 μL) with 1× Hank's Balanced Salt Solution supplemented with 2 mM MgCl₂ and 2 mM CaCl₂) (HBSS) as well as 5 mM HEPES and replenished with 100 μL of supplemented HBSS. The plate was placed in a 37° C. water bath for the reminder of the experiment and allowed to incubate for 30 min prior to beginning the experiment. Next, 100 μL of HBSS containing 80 nM 5-[1,2-³H(N)]-hydroxytryptamine creatinine sulfate ([3H]5-HT: #NET498001 MC, Lot: 3261835, Revvity) was added to the wells at a final concentration of 40 nM and incubated for 30 min to allow uptake into SERT-expressing cells. Uptake was terminated by aspiration of the wells followed by washing with supplemented HBSS (2×200 μL) and replenished with 180 μL of supplemented HBSS. Efflux was then initiated by adding 20 μL of drug (1 mM-1 μM, 1% DMSO) solution in supplemented HBSS for 20 min at a final concentration of 10 μM-1 nM (0.1% DMSO). Following the incubation, wells were washed with supplemented HBSS (3×200 μL). Cells were then lysed for 30 min by the addition of 30 μL of 10 mM NaOH. 120 μL OptiPhase HiSafe (#1200.437; Revvity) was added to the cell lysates. Counts per minute (CPM) were quantified using MicroBeta2 microplate liquid scintillation counter. Results are reported in FIG. 7A and FIG. 7B.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound, or a pharmaceutically acceptable salt thereof, having a structure of Formula I: 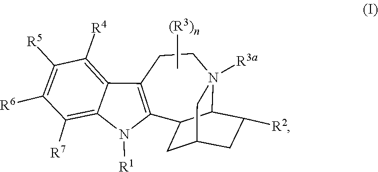
wherein:
R¹ is hydrogen or C₁₋₆ alkyl;
R² is hydrogen or C₁₋₆ alkyl;
each R³ is independently hydrogen, C₁₋₆ alkyl, C₃₋₈ cycloalkyl, or C₄₋₁₄ alkyl-cycloalkyl;
R^{3a} is absent, hydrogen or C₁₋₆ alkyl;
R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ hydroxyalkyl, C₂₋₆ alkoxyalkyl, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl, wherein at least one of R⁴, R⁵, R⁶ and R⁷ is not H;
alternatively, R⁴ and R⁵, R⁵ and R⁶, or R⁶ and R⁷ are combined with the atoms to which they are each attached to form a C₃₋₆ cycloalkyl, heterocycloalkyl, C₆₋₁₂ aryl or heteroaryl;
R^{8a}, R^{8b}, R^{8c} and R^{8d} are each independently H, C₁₋₆ alkyl; and
subscript n is 0, 1, 2, or 3;
wherein each heterocycloalkyl has 3 to 10 ring members and 1 to 4 heteroatoms each independently N, O, or S, and each heteroaryl has 5 to 10 ring members and 1 to 4 heteroatoms each independently N, O, or S,
wherein when R² is ethyl, then R⁵ is F and/or R⁷ is —OMe, and
wherein when R² is H, then R⁵ is other than H and —OMe.

2. The compound of claim 1, or a pharmaceutically acceptable salt thereof, wherein
R¹ is hydrogen.

3. The compound of claim 1, or a pharmaceutically acceptable salt thereof, wherein the compound has the structure of Formula (Ia): 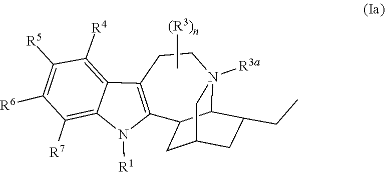
wherein R⁵ is F and/or R⁷ is —OMe.

4. The compound of claim 3, or a pharmaceutically acceptable salt thereof, wherein
R⁴, R⁵, R⁶ and R⁷ are each independently hydrogen, C₁₋₆ alkyl, halogen, C₁₋₆ haloalkyl, C₁₋₆ alkoxy, C₂₋₆ alkoxyalkyl, or C₁₋₆ haloalkoxy; and
alternatively, R⁴ and R⁵, R⁵ and R⁶, or R⁶ and R⁷ are combined with the atoms to which they are each attached to form a heterocycloalkyl having 5 to 6 ring members and 1 or 2 heteroatoms each independently O or S,
wherein at least one of R⁴, R⁵, R⁶ and R⁷ is not H, and
wherein R⁵ is F and/or R⁷ is —OMe.

5. The compound of claim 3, or a pharmaceutically acceptable salt thereof, wherein
R⁴, R⁵, R⁶ and R⁷ are each independently hydrogen, methyl, ethyl, iso-propyl, F, Cl, Br, —CF₃, —OMe, OEt, -OiPr, —CH₂OMe, or —OCF₃; and
alternatively, R⁴ and R⁵, R⁵ and R⁶, or R⁶ and R⁷ are combined with the atoms to which they are each attached to form a heterocycloalkyl having 5 ring members and 2 heteroatoms each independently O or S,
wherein at least one of R⁴, R⁵, R⁶ and R⁷ is not H, and
wherein R⁵ is F and/or R⁷ is —OMe.

6. The compound of claim 3, or a pharmaceutically acceptable salt thereof, wherein
R⁴, R⁵, R⁶ and R⁷ are each independently hydrogen, F, Cl, or —OMe; and
alternatively, R⁴ and R⁵, R⁵ and R⁶, or R⁶ and R⁷ are combined with the atoms to which they are each attached to form a heterocycloalkyl having 5 ring members and 2 heteroatoms each O,
wherein at least one of R⁴, R⁵, R⁶ and R⁷ is not H, and
wherein R⁵ is F and/or R⁷ is —OMe.

7. The compound of claim 3, or a pharmaceutically acceptable salt thereof, having the structure:

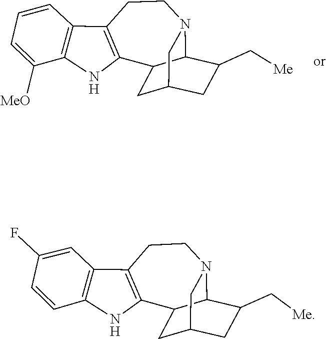 [SMILES: CCC1CC2CC3c4[nH]c5c(OC)cccc5c4CCN(C2)C13 ; CCC1CC2CC3c4[nH]c5ccc(F)cc5c4CCN(C2)C13]

8. The compound of claim 1, or a pharmaceutically acceptable salt thereof, having a structure of Formula (Ib): 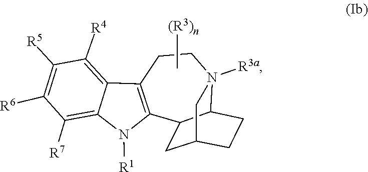
wherein
R¹ is hydrogen or C₁₋₆ alkyl;
each R³ is independently hydrogen, C₁₋₆ alkyl, C₃₋₈ cycloalkyl, or C₄₋₁₄ alkyl-cycloalkyl;
R^{3a} is absent, hydrogen or C₁₋₆ alkyl;
R⁴, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ hydroxyalkyl, C₂₋₆ alkoxyalkyl, C₁₋₆ haloalkoxy, —OR^{8a}, —NO₂, —CN, —C(O)R^{8b}, —C(O)OR^{8b}, —OC(O)R^{8b}, —OC(O)OR⁸%, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl;
R⁵ is C₁₋₆ alkyl, C₂₋₆ alkenyl, C₂₋₆ alkynyl, halogen, C₁₋₆ haloalkyl, C₁₋₆ alkylamine, C₂₋₆ alkoxy, C₁₋₆ hydroxyalkyl, C₂₋₆ alkoxyalkyl, C₁₋₆ haloalkoxy, —OH, —NO₂, —CN, —C(O)R⁸%, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, heterocycloalkyl, C₁₋₆ alkyl-heterocycloalkyl, C₆₋₁₂ aryl, C₇₋₁₈ alkyl-aryl, heteroaryl, or C₁₋₆ alkyl-heteroaryl;
alternatively, R⁴ and R⁵, R⁵ and R⁶, or R⁶ and R⁷ are combined with the atoms to which they are each attached to form a C₃₋₆ cycloalkyl, heterocycloalkyl, C₆₋₁₂ aryl or heteroaryl;
R^{8a}, R^{8b}, R^{8c} and R^{8d} are each independently H, C₁₋₆ alkyl; and
subscript n is 0, 1, 2, or 3,
wherein each heterocycloalkyl has 3 to 10 ring members and 1 to 4 heteroatoms each independently N, O, or S, and each heteroaryl has 5 to 10 ring members and 1 to 4 heteroatoms each independently N, O, or S.

9. The compound of claim 8, or a pharmaceutically acceptable salt thereof, wherein
subscript n is 0.

10. The compound of claim 8, or a pharmaceutically acceptable salt thereof, wherein
R⁴, R⁶ and R⁷ are each independently hydrogen, C₁₋₆ alkyl, halogen, C₁₋₆ haloalkyl, C₁₋₆ alkoxy, C₂₋₆ alkoxyalkyl, or C₁₋₆ haloalkoxy; and
R⁵ is C₁₋₆ alkyl, halogen, C₁₋₆ haloalkyl, C₂₋₆ alkoxy, C₂₋₆ alkoxyalkyl, or C₁₋₆ haloalkoxy;
alternatively, R⁴ and R⁵, R⁵ and R⁶, or R⁶ and R⁷ are combined with the atoms to which they are each attached to form a heterocycloalkyl having 5 to 6 ring members and 1 or 2 heteroatoms each independently O or S.

11. The compound of claim 8, or a pharmaceutically acceptable salt thereof, wherein
R⁴, R⁶ and R⁷ are each independently hydrogen, methyl, ethyl, iso-propyl, F, Cl, Br, —CF₃, —OMe, OEt, -OiPr, —CH₂OMe, or —OCF₃; and
R⁵ is methyl, ethyl, iso-propyl, F, Cl, Br, —CF₃, OEt, -OiPr, —CH₂OMe, or —OCF₃;
alternatively, R⁴ and R⁵, R⁵ and R⁶, or R⁶ and R⁷ are combined with the atoms to which they are each attached to form a heterocycloalkyl having 5 ring members and 2 heteroatoms each independently O or S.

12. The compound of claim 8, or a pharmaceutically acceptable salt thereof, wherein
R⁴, R⁶ and R⁷ are each independently hydrogen, F, Cl, or —OMe; and
R⁵ is F, or C₁;
alternatively, R⁴ and R⁵, R⁵ and R⁶, or R⁶ and R⁷ are combined with the atoms to which they are each attached to form a heterocycloalkyl having 5 ring members and 2 heteroatoms each O.

13. The compound of claim 8, or a pharmaceutically acceptable salt thereof, having the structure:

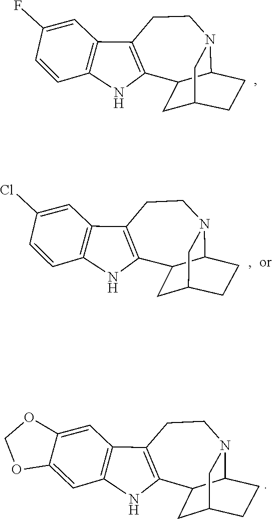 [SMILES: Fc1ccc2[nH]c3c(c2c1)CCN1CC2CCC1C3C2 ; Clc1ccc2[nH]c3c(c2c1)CCN1CC2CCC1C3C2 ; c1c2c(cc3c4c([nH]c13)C1CC3CCC1N(CC4)C3)OCO2]

14. The compound of claim 8, or a pharmaceutically acceptable salt thereof, having the structure:

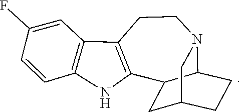 [SMILES: Fc1ccc2[nH]c3c(c2c1)CCN1CC2CCC1C3C2]

15. A pharmaceutical composition, comprising a therapeutically effective amount of a compound of claim 1, or a pharmaceutically acceptable salt thereof.

16. A method of treating a disease, comprising administering to a subject in need thereof, a therapeutically effective amount of a compound of claim 1, or a pharmaceutically acceptable salt thereof, thereby treating the disease.

17.-19. (canceled)

20. The method of claim 16, wherein the disease is depression.

21. (canceled)

22. The method of claim 16, wherein the disease is Alzheimer's disease or Parkinson's disease.

23.-34. (canceled)

35. A method for increasing neural plasticity and increasing dendritic spine density, the method comprising contacting a neuronal cell with a compound of claim 1, or a pharmaceutically acceptable salt thereof, in an amount sufficient to increase neural plasticity and increase dendritic spine density of the neuronal cell.

36. A method for modulating the function of a serotonin transporter, the method comprising contacting the serotonin transporter with a compound of claim 1, or a pharmaceutically acceptable salt thereof, in an amount sufficient to modulate the function of the serotonin transporter.

37. (canceled)
